(19) United States
(12) Patent Application Publication
(10) Pub. No.: US 2026/0128181 A1
(43) Pub. Date: May 7, 2026

(54) PROGRAM, INFORMATION PROCESSING DEVICE, METHOD, AND SYSTEM

(71) Applicant: PRECISION CO., LTD., Tokyo (JP)

(72) Inventor: Hisahiko SATO, Tokyo (JP)

(73) Assignee: PRECISION CO., LTD., Tokyo (JP)

(21) Appl. No.: 19/436,672

(22) Filed: Dec. 30, 2025

Related U.S. Application Data
(63) Continuation of application No. PCT/JP2024/022996, filed on Jun. 25, 2024.

(30) Foreign Application Priority Data
Jun. 30, 2023 (JP) 2023-107932

(51) Int. Cl. G16H 50/70; G06F 16/2457; G16H 10/60

(52) U.S. Cl. CPC G16H 50/70; G06F 16/24575; G16H 10/60

(57) ABSTRACT

An information processing apparatus including a processor and a memory storing a search database where operation instruction data used to instruct the information processing apparatus to execute a specific operation is stored is disclosed. The processor receives, as an input, at least one of the medical history information, the information input by the patient, the disease name, the symptom name or the problem in the problem list, search the search database using the input and obtain, as the operation instruction data, operation instruction data corresponding to a word or a sentence included in the input, present, on a display, the obtained operation instruction data as at least one candidate to a healthcare professional and receive an approval input from the healthcare professional, and in response to receiving the approval input, output the operation instruction data to the information processing apparatus to execute the specific operation.

### CROSS-REFERENCE TO RELATED APPLICATION

This application is a continuation of International Application No. PCT/JP2024/022996, filed Jun. 25, 2024, which claims priority to Japanese Patent Application No.2023-107932, filed Jun. 30, 2023, the entire contents of each are incorporated herein by reference.

### FIELD

The present disclosure relates to an information processing apparatus and a method.

### BACKGROUND

Electronic medical records (EMRs) are known, in which a physician electronically records the content and results of medical interviews with a patient, and further electronically records a history of medical practices performed on the patient.

As a technology related to the above-described technology, there is a technology disclosed in Japanese Unexamined Patent Application Publication No. 2013-156844.

Japanese Unexamined Patent Application Publication No. 2013-156844 discloses a technology relating to a medical support apparatus. In the medical support apparatus, input item display means displays input items on a display. Input item selection means selects one input item from a plurality of the input items. Voice recognition means performs voice recognition of input voice using a selected dictionary and extracts word candidates for the voice. Word candidate display means displays the extracted word candidates on the display. Selection operation acceptance means accepts a selection operation of one word candidate from the word candidates. Storage control means stores the one word candidate that has been selected in storage means as an answer to the selected one input item.

In the technology described in Japanese Unexamined Patent Application Publication No. 2013-156844, voice recognition processing is performed using a specialized dictionary in the medical field, but even in this case, it is necessary for healthcare professionals to perform voice input, and there has been a demand to reduce this effort.

Accordingly, the present disclosure has been made to solve the above problem, and an object thereof is to provide a technology for achieving automation of processing of medical information by healthcare professionals or semi-automation that requires approval from a user.

### SUMMARY

A program for operating a computer comprising a processor and a memory. The memory stores a search database in which operation instruction data used to instruct the computer and/or another computer to execute a specific operation is stored, or a program or a machine learning model that generates operation instruction data. The program causes the processor to execute a first step of accepting an input of at least one of medical history information, information input by a patient, a disease name, a symptom name, a problem in a problem list, and a clinical note, and a second step of searching the search database using the input accepted in the first step to obtain operation instruction data for instructing the computer and/or the other computer to execute a specific operation, or inputting the input accepted in the first step into the program or the machine learning model to obtain operation instruction data for instructing the computer and/or the other computer to execute a specific operation.

According to the present disclosure, it is possible to achieve automation of processing of medical information by healthcare professionals or semi-automation that requires approval from a user. Further, the present disclosure also semi-automates registration tasks such as document registration and order instruction registration, which are functions for efficiently using electronic medical records (EMRs), thereby enabling efficient use of EMRs.

### BRIEF DESCRIPTION OF DRAWINGS

FIG. 1 is a diagram showing an overall configuration of a system according to an embodiment.

FIG. 2 is a diagram showing a functional configuration of a terminal apparatus 10 according to an embodiment.

FIG. 3 is a diagram showing a functional configuration of a terminal apparatus 20 according to an embodiment.

FIG. 4 is a diagram showing a functional configuration of a server according to an embodiment.

FIG. 5 is a diagram showing an example of a data structure of a search database according to an embodiment.

FIG. 6 is a diagram showing another example of a data structure of a search database according to an embodiment.

FIG. 7 is a flowchart showing an example of a processing flow in a system according to an embodiment.

FIG. 8 is a flowchart showing another example of a processing flow in a system according to an embodiment.

FIG. 9 is a flowchart showing still another example of a processing flow in a system according to an embodiment.

FIG. 10 is a flowchart showing still another example of a processing flow in a system according to an embodiment.

FIG. 11 is a schematic diagram illustrating an example of a screen displayed on a terminal apparatus according to an embodiment.

FIG. 12 is a schematic diagram illustrating another example of a screen displayed on a terminal apparatus according to an embodiment.

FIG. 13 is a schematic diagram illustrating still another example of a screen displayed on a terminal apparatus according to an embodiment.

FIG. 14 is a schematic diagram illustrating still another example of a screen displayed on a terminal apparatus according to an embodiment.

FIG. 15 is a schematic diagram illustrating still another example of a screen displayed on a terminal apparatus according to an embodiment.

FIG. 16 is a schematic diagram illustrating still another example of a screen displayed on a terminal apparatus according to an embodiment.

FIG. 17 is a schematic diagram illustrating still another example of a screen displayed on a terminal apparatus according to an embodiment.

FIG. 18 is a schematic diagram illustrating still another example of a screen displayed on a terminal apparatus according to an embodiment.

FIG. 19 is a schematic diagram illustrating still another example of a screen displayed on a terminal apparatus according to an embodiment.

FIG. 20 is a schematic diagram illustrating still another example of a screen displayed on a terminal apparatus according to an embodiment.

FIG. 21 is a schematic diagram illustrating still another example of a screen displayed on a terminal apparatus according to an embodiment.

FIG. 22 is a schematic diagram illustrating still another example of a screen displayed on a terminal apparatus according to an embodiment.

FIG. 23 is a schematic diagram illustrating still another example of a screen displayed on a terminal apparatus according to an embodiment.

FIG. 24 is a schematic diagram illustrating still another example of a screen displayed on a terminal apparatus according to an embodiment.

FIG. 25 is a schematic diagram illustrating still another example of a screen displayed on a terminal apparatus according to an embodiment.

FIG. 26 is a schematic diagram illustrating still another example of a screen displayed on a terminal apparatus according to an embodiment.

FIG. 27 is a schematic diagram illustrating still another example of a screen displayed on a terminal apparatus according to an embodiment.

FIG. 28 is a schematic diagram illustrating still another example of a screen displayed on a terminal apparatus according to an embodiment.

### DETAILED DESCRIPTION

Hereinafter, embodiments of the present disclosure will be described with reference to the drawings. In all the drawings for describing the embodiments, common constituent elements are denoted by the same reference numerals, and repetitive descriptions thereof will be omitted. The following embodiments do not unduly limit the contents of the present disclosure described in the claims. Furthermore, not all the constituent elements shown in the embodiments are necessarily essential constituent elements of the present disclosure. Moreover, each drawing is a schematic diagram and is not necessarily illustrated strictly.

In the following description, “processor” refers to one or more processors. At least one processor is typically a microprocessor such as a CPU (Central Processing Unit), but may be another type of processor such as a GPU (Graphics Processing Unit). At least one processor may be single-core or multi-core.

Furthermore, at least one processor may be a processor in a broad sense, such as a hardware circuit (for example, an FPGA (Field-Programmable Gate Array) or an ASIC (Application Specific Integrated Circuit)) that performs part or all of the processing.

In the following description, information from which an output is obtained in response to an input may be described using an expression such as “xxx table”, but this information may be data of any structure or may be a learning model such as a neural network that generates an output in response to an input. Therefore, “xxx table” can be referred to as “xxx information”.

In the following description, the configuration of each table is an example, and one table may be divided into two or more tables, or all or part of two or more tables may be one table.

In the following description, processing may be described with “program” as the subject, but since a program performs defined processing while appropriately using a storage unit and/or an interface unit, etc., by being executed by a processor, the subject of the processing may be the processor (or a device such as a controller having the processor).

A program may be installed in a device such as a computer, or may be, for example, in a program distribution server or a computer-readable (for example, non-transitory) recording medium. In the following description, two or more programs may be implemented as one program, or one program may be implemented as two or more programs.

In the following description, identification numbers are used as identification information for various objects, but other types of identification information (for example, identifiers including alphabetic characters or symbols) may be employed.

In the following description, when elements of the same type are described without distinction, a reference sign (or a common sign among reference signs) is used, and when elements of the same type are described with distinction, an identification number (or reference sign) of the element may be used.

In the following description, control lines and information lines indicate those considered necessary for the explanation, and do not necessarily indicate all control lines and information lines in the product. All configurations may be interconnected with each other.

### 0 System Overview

The system according to the present disclosure is a system in which healthcare professionals, represented by physicians, issue instructions for operation execution in a computer-interpretable format to a computer in this system or in another system, triggered by the input of medical information such as medical history information and clinical notes. In addition, the system is configured to issue further instructions for operation execution in a computer-interpretable format to the computer, triggered by a response of results from the computer based on the instructions, and to repeat the above operations. The medical history includes records of a patient's past medical history, allergy history, family history, history of present illness (current symptoms, progress, examination findings, treatment), etc., and is part of EMR data. In many cases, it may be described in natural language, but the data may also be standardized using the form of an EMR template. In particular, in templates, past medical history, family history, and history of present illness may be recorded in separate template formats, and during natural language processing as described below, the two or three may be treated as different inputs to improve accuracy.

Hereinafter, the outline of the system according to the present disclosure will be described, but the system according to the present disclosure need not be interpreted in a limited manner based on the following description, and the contents of the present disclosure should be understood based on the disclosure of this specification and the ordinary technical knowledge and common sense possessed by those skilled in the art.

The system according to the present disclosure takes as input natural language text of medical history information including time information, and obtains data for instructing a machine (computer) according to a format (hereinafter, machine-oriented operation instruction data) by generating it using similarity search with natural language text in a search database of machine-oriented operation instruction data associated with natural language text, words, or tags, or using a generation model or machine processing of machine-oriented operation instruction data, and reduces the effort of human machine input by issuing operation instructions to the machine.

Furthermore, the system according to the present disclosure updates the medical history by converting results of or responses to operation instructions to the machine into text together with time information, and continuously reduces the effort of instructing the machine by repeating the acquisition of data for operation instructions to the machine according to the format from the second time onward.

At this time, by combining the natural language of the medical history, the natural language that guides healthcare professionals to operation instruction data to the machine, and the function of obtaining machine-oriented operation instruction data based on the two natural languages, it is possible to simultaneously achieve addition to the medical history, navigation of operation instructions to the machine having a chat function, and labor saving in inputting operation instructions to the machine.

Furthermore, the system according to the present disclosure comprises a user interface for inputting medical history, and a user interface for obtaining operation instruction data to the machine obtained by using the medical history as input and using dedicated search or dedicated generation AI (artificial intelligence), displaying operation instructions to the machine, and obtaining approval. The operation instructions to the machine in this case include natural language addition operation instructions, questionnaire display operation instructions, examination order operation instructions, prescription operation instructions, medical fee disease name input operation instructions, patient message sending operation instructions, RPA operation instructions, template display operation instructions, template data input operation instructions, filtered examination result display operation instructions, filtered prescription information display operation instructions, appointment reservation operation instructions, and the like. Hereinafter, operation instruction data to the machine includes these instructions.

In particular, the system according to the present disclosure comprises a user interface for inputting medical history, and a user interface for obtaining a plurality of instructions to the machine obtained by using the medical history as input and using search, displaying operation instructions to the machine, and selecting or modifying dates or the like to update to new operation instructions.

Furthermore, the system according to the present disclosure comprises a user interface for inputting medical history, and a user interface for obtaining one or more instructions to the machine obtained by using the medical history as input and using search, and displaying and selecting operation instructions to the machine, and has a user interface for displaying and modifying operation instructions to the machine and their tags.

Furthermore, the system according to the present disclosure comprises a screen for inputting medical history, and when operation instructions to the machine obtained by using search with the medical history as input or a generation model dedicated to operation instructions to the machine are obtained, and operation instructions to the machine corresponding to the medical history are displayed on the same screen as search word candidates and a button for obtaining approval is pressed, the content of the instructions is added to the medical history, or as a result of approval of the instructions, the medical history is updated by receiving results input by the patient to a questionnaire displayed on another machine or by obtaining examination results as a result of performing an examination, and candidate updates of operation instructions to the next machine are displayed, and a user interface for approving the candidate updates, and the operation of updating the medical history updated as a result of the approval is repeated.

When obtaining this approval, it is also possible to display the difference between the current patient and the patient for whom this instruction was given. In one example, when searching for similar cases for a patient with cough and nasal discharge in a prescription for an influenza patient and obtaining operation instruction data, if a patient with cough, nasal discharge, and fever is found as a search result, natural language processing may be performed to display the difference in an easy-to-understand manner in the symptom column of the template display, such as current patient's medical history: cough (+), nasal discharge (+), fever (−), original medical history of operation instruction data: cough (+), nasal discharge (+), fever (+), by indicating the presence or absence of symptoms with plus and minus signs. Furthermore, to make this information easier to compare and understand, chronological information and comparisons may be displayed in a table format.

In particular, the system according to the present disclosure comprises a function for inputting medical history, and a user interface for obtaining operation instructions to the machine obtained by using search with the medical history as input or a generation model dedicated to operation instructions to the machine, displaying operation instructions to the machine, and obtaining approval, and is accompanied by a function for displaying natural language that guides healthcare professionals to operation instruction data to the machine based on the medical history.

When updating the medical history, the medical history update portion may be highlighted for the user, and an interface may be provided that records that the updater of the medical history is natural language processing, manages the change history as a history, and displays differences. In particular, when the medical history is described using a template, and template values such as history of present illness, past medical history, family history, presence or absence of allergies, etc., are updated based on the input content of the questionnaire, the updated portion may be highlighted to indicate to the user that information has been added or updated.

Furthermore, the system according to the present disclosure is a system comprising a user interface for inputting medical history, wherein a problem list is updated based on natural language text described in the interface for describing the medical history, search words are set based on the problem list, and past examinations, past prescriptions, and past machine instructions are searched.

Furthermore, the system according to the present disclosure is a system comprising a user interface for inputting medical history, wherein chat text is generated based on natural language text described in the interface for describing the medical history, search words are set, and past examinations, past prescriptions, and past machine instructions are searched.

Furthermore, the system according to the present disclosure comprises data in which at least medical history or data generated from the medical history and medical instruction information or medical history addition instruction information are stored in pairs, and when input of medical history is accepted, search words are generated based on this medical history, medical instruction information is searched based on the generated search words, and the medical instruction information is presented to the user. Furthermore, the frequency of medical instruction information or the name or title of a user who has given medical instructions in the past may be displayed.

### One Embodiment

### 1 Configuration Diagram of Entire System

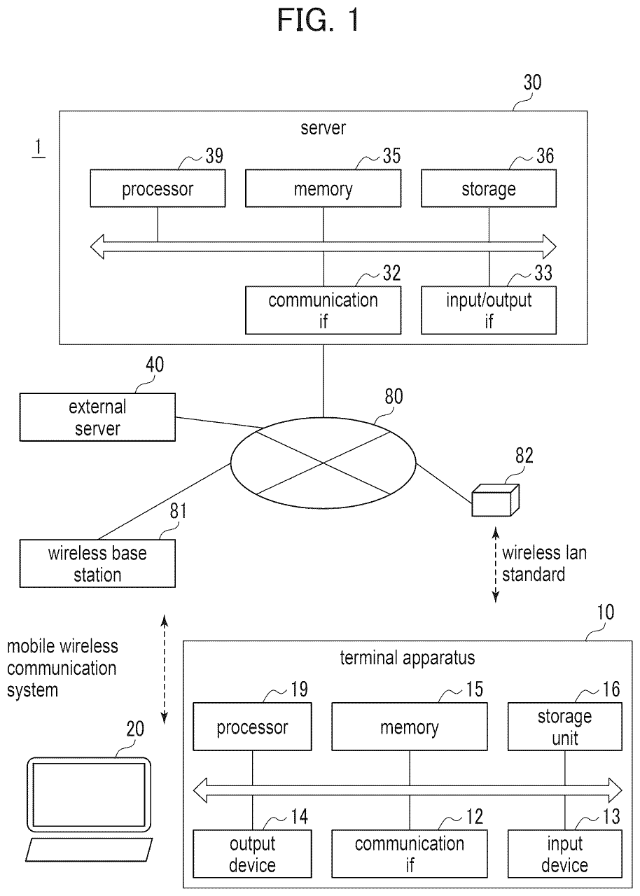

FIG. 1 is a diagram showing the overall configuration of a medical support system (hereinafter simply referred to as “system”) 1 according to the present embodiment. As shown in FIG. 1, the system 1 includes a plurality of terminal apparatuses (FIG. 1 shows a terminal apparatus 10 and a terminal apparatus 20, which may be collectively referred to as “terminal apparatus 10” hereinafter) and a server 30. The terminal apparatuses 10 and 20 and the server 30 are connected to each other via a network 80 so as to be capable of communicating with each other. The network 80 is configured by a wired or wireless network. In the present embodiment, the server 30 is a server having a function as a Web server (including a cloud server), and exchanges information with the terminal apparatuses 10 and 20 via Web pages. Furthermore, although a Web page browser for browsing Web pages is installed in the terminal apparatuses 10 and 20, a dedicated application for providing services of the server 30 may be installed and configured to be browsable by the dedicated application.

Since the hardware configuration of the terminal apparatus 10 and the hardware configuration of the terminal apparatus 20 are common, the description of the hardware configuration of the terminal apparatus 20 will be omitted by describing the hardware configuration of the terminal apparatus 10. For components of the terminal apparatus 20 that are the same as components of the terminal apparatus 10, the reference signs start with 2, and the system of reference signs is the same.

The terminal apparatus 10 is an apparatus operated by a patient. The patient browses questionnaire questions of an electronic questionnaire displayed on the output device 14 of the terminal apparatus 10, and inputs questionnaire response content corresponding to the questionnaire questions using the input device 13. A healthcare professional may support the patient's operation of the terminal apparatus 10, that is, the input of questionnaire response content of the electronic questionnaire. The terminal apparatus 10 is realized by a stationary PC (Personal Computer), a laptop PC, or the like. In addition, the terminal apparatus 10 may be, for example, a tablet compatible with a mobile communication system, or a mobile terminal such as a smartphone.

The terminal apparatus 10 is connected to the server 30 via the network 80 so as to be capable of communicating therewith. The terminal apparatus 10 is connected to the network 80 by communicating with communication devices such as a wireless base station 81 compatible with communication standards such as 4G, 5G, and LTE (Long Term Evolution), and a wireless LAN router 82 compatible with wireless LAN (Local Area Network) standards such as IEEE (Institute of Electrical and Electronics Engineers) 802.11. As shown in FIG. 1, the terminal apparatus 10 comprises a communication IF (Interface) 12, an input device 13, an output device 14, a memory 15, a storage unit 16, and a processor 19.

The communication IF 12 is an interface for inputting and outputting signals for the terminal apparatus 10 to communicate with external devices. The input device 13 is an input device (for example, a keyboard, a touch panel, a touch pad, a pointing device such as a mouse, etc.) for accepting input operations from the user. The output device 14 is an output device (display, speaker, etc.) for presenting information to the user. The memory 15 is for temporarily storing programs and data processed by the programs, etc., and is, for example, a volatile memory such as a DRAM (Dynamic Random Access Memory). The storage unit 16 is a storage device for storing data, and is, for example, a flash memory or an HDD (Hard Disc Drive). The processor 19 is hardware for executing an instruction set described in a program, and is configured by an arithmetic device, registers, peripheral circuits, and the like.

The terminal apparatus 20 is an apparatus operated by a healthcare professional or an administrator of the system 1. Here, healthcare professional is a concept that includes physicians, nurses, laboratory technicians having medical knowledge, and the like. In the following description, unless healthcare professionals and administrators of the system 1 are described separately, healthcare professionals shall include administrators of the system 1.

The healthcare professional browses medical information using the terminal apparatus 20 and inputs instructions to the patient including medical treatment. The input instructions are processed in the server 30 described later, or processed in an external server 40 outside the system 1, and the processing results are displayed on the terminal apparatus 20. Furthermore, the input results of the electronic questionnaire input by the patient via the terminal apparatus 10 are also displayed on the terminal apparatus 20.

The server 30 is managed by an administrator of the system 1 of the present embodiment, and the stored content is appropriately corrected/added/deleted by healthcare professionals who are users of the terminal apparatus 20.

The server 30 is also an EMR apparatus, and healthcare professionals in a medical facility browse input items and input content of the EMR via the terminal apparatus 20, and correct/add to the input content. At least a part of logs of various instruction information including medical instruction information, which is also a feature of the system according to the present disclosure, is reflected as input content of the EMR.

The server 30 is a computer connected to the network 80. The server 30 comprises a communication IF 32, an input/output IF 33, a memory 35, a storage 36, and a processor 39.

The communication IF 32 is an interface for inputting and outputting signals for the server 30 to communicate with external devices. The input/output IF 33 functions as an interface with an input device for accepting input operations from the user and an output device for presenting information to the user. The memory 35 is for temporarily storing programs and data processed by the programs, etc., and is, for example, a volatile memory such as a DRAM (Dynamic Random Access Memory). The storage 36 is a storage device for storing data, and is, for example, a flash memory or an HDD (Hard Disc Drive). The processor 39 is hardware for executing an instruction set described in a program, and is configured by an arithmetic device, registers, peripheral circuits, and the like.

### 1.1 Functional Configuration of Terminal Apparatus 10

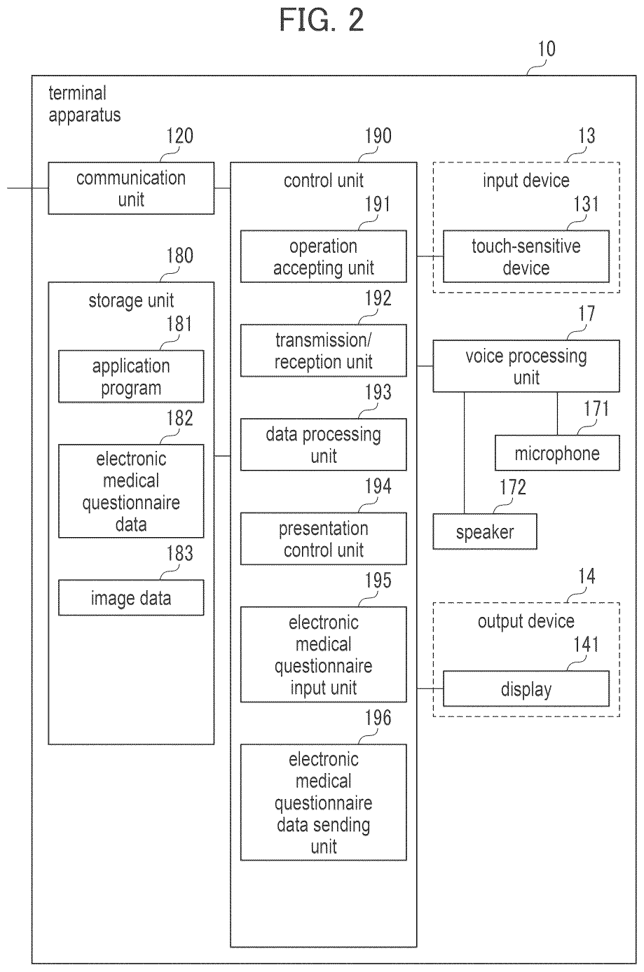

FIG. 2 is a block diagram showing an example of a functional configuration of the terminal apparatus 10 shown in FIG. 1. The terminal apparatus 10 shown in FIG. 2 is realized by, for example, a PC, a mobile terminal, or a wearable terminal. As shown in FIG. 2, the terminal apparatus 10 comprises a communication unit 120, an input device 13, an output device 14, a voice processing unit 17, a microphone 171, a speaker 172, a storage unit 180, and a control unit 190. Each block included in the terminal apparatus 10 is electrically connected by, for example, a bus or the like.

The communication unit 120 performs processing such as modulation and demodulation processing for the terminal apparatus 10 to communicate with other devices. The communication unit 120 performs transmission processing on the signal generated by the control unit 190 and transmits the signal to the outside (for example, the server 30). The communication unit 120 performs reception processing on the signal received from the outside and outputs the signal to the control unit 190.

The input device 13 is a device for a user operating the terminal apparatus 10 to input instructions or information. The input device 13 may be realized by, for example, a keyboard, a mouse, a reader, or the like. When the terminal apparatus 10 is a mobile terminal or the like, it is realized by a touch-sensitive device 131 or the like into which instructions are input by touching an operation surface. The input device 13 converts instructions input from the user into electrical signals and outputs the electrical signals to the control unit 190. The input device 13 may include, for example, a receiving port that accepts electrical signals input from an external input device.

The output device 14 is a device for presenting information to the user operating the terminal apparatus 10. The output device 14 is realized by, for example, a display 141 or the like. The display 141 displays data according to control by the control unit 190. The display 141 is realized by, for example, an LCD (Liquid Crystal Display), an organic EL (Electro-Luminescence) display, or the like.

The voice processing unit 17 performs, for example, digital-to-analog conversion processing of voice signals.

The voice processing unit 17 converts the signal supplied from the microphone 171 into a digital signal and supplies the converted signal to the control unit 190. Furthermore, the voice processing unit 17 supplies a voice signal to the speaker 172. The voice processing unit 17 is realized by, for example, a processor for voice processing. The microphone 171 accepts voice input and supplies a voice signal corresponding to the voice input to the voice processing unit 17. The speaker 172 converts the voice signal supplied from the voice processing unit 17 into voice and outputs the voice to the outside of the terminal apparatus 10.

The storage unit 180 is realized by, for example, the memory 15, the storage unit 16, and the like, and stores data and programs used by the terminal apparatus 10. The storage unit 180 stores, for example, electronic questionnaire data 182 and image data 183.

The electronic questionnaire data 182 is data of an electronic questionnaire generated based on electronic questionnaire data 3028 stored in the server 30, in which the patient operates the terminal apparatus 10 to input questionnaire response content.

The image data 183 is image data used when presenting the electronic questionnaire to the patient.

The control unit 190 is realized by the processor 19 reading an application program 181 stored in the storage unit 180 and executing instructions included in the application program 181. The control unit 190 controls the operation of the terminal apparatus 10. The control unit 190 operates in accordance with the application program 181 stored in the storage unit 180, thereby exhibiting functions as an operation accepting unit 191, a transmitting and receiving unit 192, a data processing unit 193, a presentation control unit 194, an electronic questionnaire input unit 195, and an electronic questionnaire data sending unit 196.

The operation accepting unit 191 performs processing for accepting instructions or information input from the input device 13. Specifically, for example, the operation accepting unit 191 accepts information based on instructions input from a keyboard, a mouse, or the like.

The transmitting and receiving unit 192 performs processing for the terminal apparatus 10 to transmit and receive data to and from external devices such as the server 30 in accordance with a communication protocol.

Specifically, for example, the transmitting and receiving unit 192 transmits work content input from the user to the server 30. Furthermore, the transmitting and receiving unit 192 receives information related to the user from the server 30.

The data processing unit 193 performs processing of performing calculations on data for which the terminal apparatus 10 has accepted input in accordance with the application program 181, and outputting the calculation results to the memory 15 or the like.

The presentation control unit 194 controls the output device 14 to present information provided from the server 30 to the user. Specifically, for example, the presentation control unit 194 causes the display 141 to display information transmitted from the server 30. Furthermore, the presentation control unit 194 causes the speaker 172 to output information transmitted from the server 30.

The electronic questionnaire input unit 195 accepts input of questionnaire response content input by the patient for questionnaire questions of the electronic questionnaire displayed on the display 141 by the presentation control unit 194 (the input of this questionnaire response content includes selection input for any of the options of preset questionnaire questions), and stores the accepted input of questionnaire response content in the electronic questionnaire data 182 by associating it with questionnaire items associated with this questionnaire response content.

The electronic questionnaire data sending unit 196 sends to the server 30 the electronic questionnaire data 182 for which input of questionnaire response content has been accepted by the electronic questionnaire input unit 195 and which has been stored as electronic questionnaire data 182 in the storage unit 180.

### 1.2 Functional Configuration of Terminal Apparatus 20

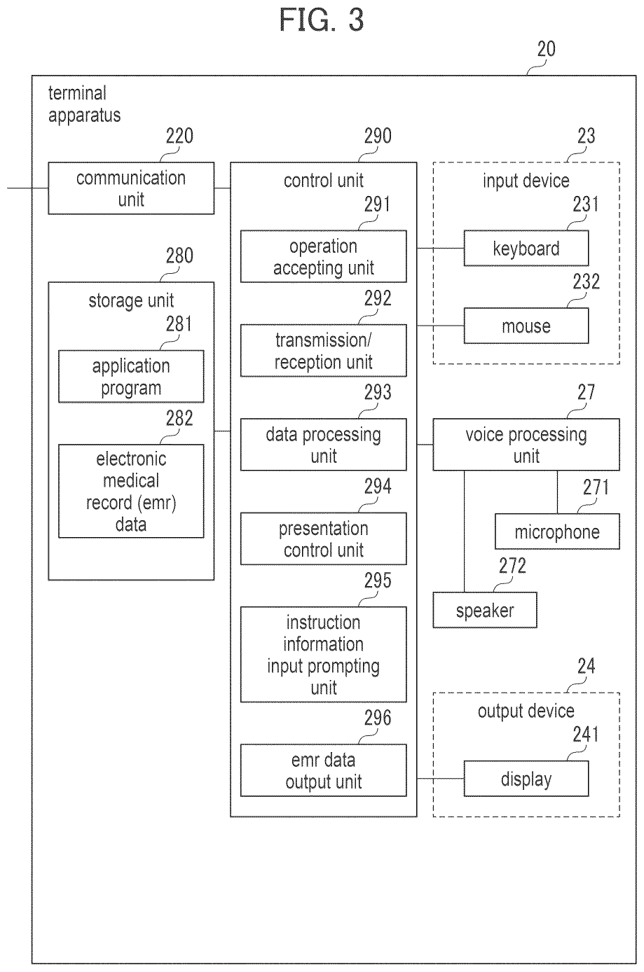

FIG. 3 is a block diagram illustrating an example of a functional configuration of the terminal apparatus 20 shown in FIG. 1. The terminal apparatus 10 shown in FIG. 3 is realized by, for example, a PC, a portable terminal, or a wearable terminal.

Since the functional configuration of the terminal apparatus 20 has much in common with the functional configuration of the terminal apparatus 10, the description will focus on the parts that differ from the functional configuration of the terminal apparatus 10.

The storage unit 280 is realized by, for example, the memory 25 and the storage 26, and stores data and programs used by the terminal apparatus 20. The storage unit 280 stores, for example, electronic medical record data 282.

The electronic medical record data 282 is data input by a healthcare professional operating the terminal apparatus 20, and at least part of a log of various instruction information including medical instruction information generated by each functional unit of the server 30, which will be described later, is reflected as input content of the electronic medical record.

The control unit 290 is realized by the processor 29 reading an application program 281 stored in the storage unit 280 and executing instructions included in the application program 281. The control unit 290 controls the operation of the terminal apparatus 20. The control unit 290 operates according to the application program 281 stored in the storage unit 280 to perform functions as an operation accepting unit 291, a transmitting and receiving unit 292, a data processing unit 293, a presentation control unit 294, an instruction information input instruction unit 295, and an electronic medical record data output unit 296.

The operations of the operation accepting unit 291, the transmitting and receiving unit 292, the data processing unit 293, and the presentation control unit 294 are the same as those of the operation accepting unit 191, the transmitting and receiving unit 192, the data processing unit 193, and the presentation control unit 194 of the terminal apparatus 10, and thus descriptions thereof are omitted.

The instruction information input instruction unit 295 performs operations such as approving and selecting various instructions to other computers including the natural language generation model 3023 and the like, based on various instruction information including medical instruction information from the natural language generation model 3023 and the like provided in the server 30, and gives various instructions to modules in the server 30 or modules in the external server 40. Details of the medical instruction information and the various instruction information will be described later.

The electronic medical record data output unit 296 outputs, to the server 30 as electronic medical record data, information including various instruction information from the instruction information input instruction unit 295 to the server 30 and the like, and output from the server 30 and the like based on the various instruction information.

### 1.3 Functional Configuration of Server 30

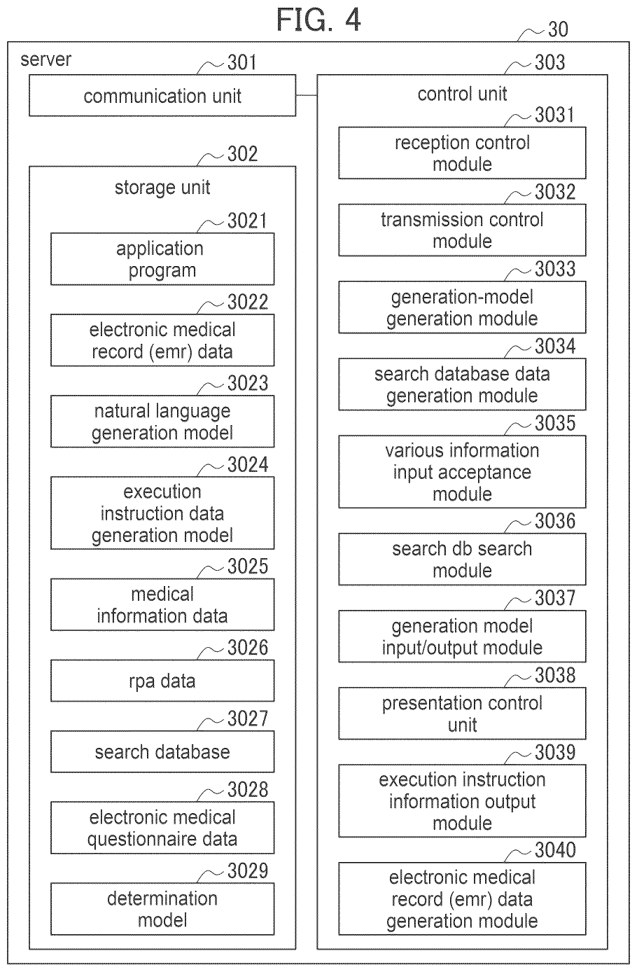

FIG. 4 is a diagram illustrating an example of a functional configuration of the server 30. As shown in FIG. 4, the server 30 performs functions as a communication unit 301, a storage unit 302, and a control unit 303.

The communication unit 301 performs processing for the server 30 to communicate with external apparatuses.

The storage unit 302 comprises, for example, EMR data 3022, a natural language generation model 3023, an operation instruction data generation model 3024, medical information data 3025, RPA data 3026, a search database 3027, electronic medical questionnaire data 3028, a determination model 3029, and the like.

The EMR data 3022 is EMR data 3022 for patients who have visited a medical facility that uses the server 30. Since the EMR data 3022 itself is known, the details thereof are omitted, but it generally includes medical history information with time information, patient examination data, prescription data for the patient, and the like. The EMR data 3022 comprises input items and input content associated with the input items. Although there is no particular limitation on the data format of the EMR data 3022, the EMR data 3022 in the present embodiment is obtained by converting data described in XAML (Extensible Application Markup Language) into JSON (JavaScript Object Notation) (JavaScript is a registered trademark) format.

The EMR data 3022 is preferably configured such that identifiers such as numeric strings are assigned to the input items, and these identifiers also constitute the EMR data 3022.

In the system 1 of the present embodiment, the EMR data 3022 is an example of medical information. In addition, as described later, logs of communications between the server 30 and the terminal apparatus 20, and communications between modules in the server 30 and the external server 40, are also incorporated as EMR data 3022 after receiving approval from the healthcare professional operating the terminal apparatus 20. Furthermore, clinical notes input by a healthcare professional via the terminal apparatus 20 are also incorporated as EMR data 3022 after receiving approval from the healthcare professional operating the terminal apparatus 20.

The natural language generation model 3023, as represented by ChatGPT, for example, is configured to output natural language in response to input natural language. In one example, fine-tuning may be performed to output natural language that explains why operation instruction data for a machine is being presented to a healthcare professional (that is, the reason for presenting the operation instruction data), and the user experience can be improved by displaying this near a button for inputting approval of the operation instruction data. A natural language model that generates such natural language can be created by preparing a list of information consisting of three pairs: medical history information, machine instruction information, and “natural language that explains why operation instruction data for a machine is being presented to a healthcare professional,” and performing machine learning to generate “natural language that explains why operation instruction data for a machine presented to a healthcare professional is being presented” by using the medical history information and machine instruction information as inputs.

Since the chat function and large language models themselves are known technologies, a description of their specific configuration and generation methods is omitted here.

The operation instruction data generation model 3024, similarly to the natural language generation model 3023, is generated by being trained in advance using appropriate natural language information so as to be capable of returning appropriate responses to specific medical instruction information and the like.

The operation instruction data generation model 3024 in the system 1 of the present embodiment includes an electronic medical questionnaire data generation model that generates electronic medical questionnaire data, an examination order instruction data generation model that generates appropriate examination orders for a specific patient based on the patient's past medical history, medical interview results, and further past examination content, prescription content, and disease name registration content, a prescription order instruction data generation model that generates appropriate prescription orders for a specific patient, a disease name registration instruction generation model that generates appropriate disease name registration instructions for a specific patient, an RPA operation instruction data generation model that generates appropriate RPA (Robotic Process Automation) orders for modules of the server 30 or the external server 40, and the like. Here, the RPA orders include, for example, instructions to a module that performs in-facility announcements at a medical facility, a module that generates and transmits or displays guidance messages to patients within the medical facility, and the like, and generate operation instructions that cause these modules to perform appropriate operations.

The operation instruction data generation model 3024 is preferably operation instruction data used to instruct a module to perform a specific operation, in a format that can be interpreted by the modules in the server 30 and the external server 40.

Since the method for generating the natural language generation model 3023 is known, an explanation thereof is omitted here, but since the operation instruction data generation model 3024 is, in a sense, a natural language generation model customized for medical instruction information, the method for generating the same will be described later.

The medical information data 3025 is data relating to medical information received by various information input reception modules 3035 described later. Typical examples of the medical information referred to here include medication record OCR results, referral letter OCR results, clinical notes, text or voice information input by patients, EMR logs, communication records, print instructions, logistics information, attendance information of healthcare professionals, electronic textbooks, drug interaction information, contraindication state information, and information existing in medical settings. Generally, medical information is a broader concept than electronic medical records, and a part of the medical information data 3025 may overlap with the EMR data 3022.

It should be noted that clinical notes are text written by healthcare professionals at clinical sites that do not necessarily bear official responsibility.

Specifically, they are notes of ideas that come to mind, notes from conferences, and the like. The natural language generation model 3023 and the like can automatically create clinical notes. However, in order to save them as an EMR, it is necessary for a physician to approve the clinical notes. In addition, the contents written on paper medical questionnaires, progress records brought by patients, and the like are often treated as clinical notes in actual medical settings, and become EMR data 3022 when approved by a physician.

The RPA data 3026 is RPA data for modules in the server 30 or the external server 40.

The search database (DB: Database) 3027 is a database showing a correspondence relationship between a word or a sequence of words included in medical information, or a search tag created by performing natural language processing on the basis of medical information, and operation instruction data for instructing a module in the server 30 or the external server 40 to execute a specific operation corresponding to this search tag, or a natural language sentence to be input to the natural language generation model 3023 or the operation instruction data generation model 3024. Details will be described later.

The electronic medical questionnaire data 3028 is data that forms the basis of an electronic medical questionnaire on which a patient performs input via the terminal apparatus 10, and data regarding content input by the patient on the basis of this data.

Preferably, the electronic medical questionnaire data 3028 is operation instruction data that defines medical interview items and medical interview questions, medical interview question types, and medical interview options associated with the medical interview items. Although there is no particular limitation on the data format of the electronic medical questionnaire data 3028, similarly to the EMR data 3022, it is obtained by converting data described in XAML into JSON format. Similarly to the EMR data 3022, the electronic medical questionnaire data 3028 is preferably configured such that identifiers such as numeric strings are assigned to the input items thereof, and these identifiers also constitute the electronic medical questionnaire data 3028.

The determination model 3029 is a model for determining whether or not to generate operation instruction data for instructing a predetermined operation to modules in the server 30 and the external server 40, in response to medical information received by various information input reception modules 3035 described later. The determination model 3029 is, for example, a so-called machine learning model. A method for generating the determination model 3029 will be described later.

The machine learning model according to the present embodiment is, for example, a composite function with parameters in which a plurality of functions are combined. A composite function with parameters is defined by a combination of a plurality of adjustable functions and parameters. The prediction model according to the present embodiment may be any composite function with parameters that satisfies the above requirements, but is assumed to be a multilayer network model (hereinafter referred to as a multilayer network). A prediction model using a multilayer network comprises an input layer, an output layer, and at least one intermediate layer or hidden layer provided between the input layer and the output layer. The prediction model is assumed to be used as a program module that is part of artificial intelligence software.

As the multilayer network according to the present embodiment, for example, a Deep Neural Network (DNN), which is a multilayer neural network subject to deep learning, may be used. As the DNN, for example, a Convolutional Neural Network (CNN) for images may be used.

Furthermore, the above is merely an example of a prediction model, and the prediction model may comprise other configurations. For example, the prediction model may be a rule-based model in which hunting information and environmental information are used as variables, and each variable is described by a function to which a coefficient derived from past results is assigned.

The control unit 303 is realized by the processor 29 reading the application program 3021 stored in the storage unit 302 and executing instructions included in the application program 3021. By operating in accordance with the application program 3021, the control unit 303 exhibits functions shown as a reception control module 3031, a transmission control module 3032, a generation model generation module 3033, a search database data generation module 3034, various information input reception modules 3035, a search DB search module 3036, a generation model input/output module 3037, a presentation control unit 3038, an operation instruction information output module 3039, and an EMR data generation module 3040.

The reception control module 3031 controls processing in which the server 30 receives a signal from an external apparatus in accordance with a communication protocol.

The transmission control module 3032 controls processing in which the server 30 transmits a signal to an external apparatus in accordance with a communication protocol.

The generation model generation module 3033 generates the operation instruction data generation model 3024 and the determination model 3029 stored in the storage unit 302 of the server 30. A specific method for generating the operation instruction data generation model 3024 and the determination model 3029 by the generation model generation module 3033 will be described later.

The search database data generation module 3034 generates data for the search database 3027 stored in the storage unit 302 of the server 30.

More specifically, the search database data generation module 3034 extracts at least one of medical history information, information input by a patient, disease names, symptom names, problems in a problem list, and clinical notes (hereinafter simply referred to as “medical information” or “medical history information”) included in the EMR data 3022 or the medical information data 3025, and records operation instruction data for a machine that was performed on the same day as or simultaneously with this medical history information or intentionally associated with the medical history, in pairs. Further, a word or a sequence of words included in the medical history information, or an expression obtained by converting the medical history information by natural language processing, is set as a search tag. Which word or sequence of words is set as a search tag is arbitrary, but examples include setting medical history, disease names, and symptom names included in medical information as search tags or display titles. Such search tags or display titles may be extracted from disease names, symptom names, and problems in the problem list included in the medical history information of the EMR data 3022. Furthermore, for extracting problems from medical history information, determination may be made on the basis of the length of one line, the presence or absence of an identifier, the presence or absence of disease names and symptom names, the presence or absence of particles, and the like. In addition, when registering disease names, symptom names, and problem lists as search tags or titles, the disease names and symptom names may be consolidated and normalized to standard disease names and standard symptom names, or typographical errors may be corrected. Furthermore, date information such as March 1 may be changed to hospitalization or examination forms and relative dates, such as the first day of hospitalization, the first day of outpatient visits, or the third day after surgery, and used as search tags or display titles.

The search database 3027 stores past medical history information and its input date and time, and further past operation instruction data generated by the search database data generation module 3034 on the basis of this past medical history information and a generation unit thereof. The search database data generation module 3034 may generate operation instruction data on the basis of operation instruction data that has temporal relevance to the generation of a search tag (for example, the same day, the same month) or that was instructed to be executed in association therewith, and store the operation instruction data in the search database 3027.

Here, a problem list is a list that shows what is being treated and what is being attempted to be resolved for the patient, and needs to be updated as appropriate each time a problem is resolved or each time a new problem appears.

When a healthcare professional describes a problem in a problem list as a medical history in the EMR data 3022 or the like, a specific symbol (for example, # or a line break symbol) may be added before the medical history, disease name, symptom name, or the like. The search database data generation module 3034 extracts this specific symbol from the EMR data 3022 or the like, determines that a word or a sequence of words following the specific symbol is a problem, and sets this word or the like as a search tag.

Then, for each search tag, the search database data generation module 3034 generates operation instruction data for instructing a module in the server 30 or the external server 40 to execute a specific operation from the EMR data or the medical information data, associates the operation instruction data with the medical history information, and stores the operation instruction data as data in the search database 3027.

The association between search tags and operation instruction data or the like may be performed manually by a healthcare professional, but the search database data generation module 3034 may analyze the EMR data 3022 or the like, learn what instructions the healthcare professional gave when a search tag appeared, and perform the association. Furthermore, when operation instruction data is associated with a plurality of tags such as #hypertension and #constipation, the operation instruction data may be divided in detail, separated on the basis of pharmaceutical efficacy information, electronic textbook information, and data associated with other tags to determine whether each is associated with #hypertension or #constipation, and stored after executing an algorithm for associating subdivided operation instruction data with each tag. Specifically, as a prescription example associated with #hypertension and #constipation, when an antihypertensive drug and a laxative are associated, the antihypertensive drug may be organized and associated with #hypertension, and the laxative may be organized and associated with #constipation. Furthermore, a drug associated with #hypertension in another association may be determined to be a therapeutic drug for hypertension, and drugs other than that may be determined to be remaining laxatives. In addition, EMR log information, search behavior logs of healthcare professionals, and sensor log information may be held in the medical information data 3025, and tags may be generated on the basis of search words described in logs of simultaneously searching electronic textbooks or the like. Furthermore, operation instruction data may be automatically generated from EMR log information and recorded in the search database 3027.

The operation instruction data associated with a search tag can include execution of a specific operation instructed to a module in the server 30 or the external server 40 by this operation instruction data. That is, when it is desired to execute a series of operations for one search tag, individual operation instruction data instructing execution of individual operations may be combined into one operation instruction data, and these may be associated and stored in the search database 3027. The series of operation executions referred to here may be operation executions performed simultaneously, or may be operation executions sequentially executed within a predetermined time period.

Furthermore, the search database data generation module 3034 presents the search tags generated by the search database data generation module 3034 via the display 241 of the terminal apparatus 20, and accepts correction instructions for the search tags from a user via the input device 23 and the voice processing unit 27 of the terminal apparatus 20. The search database data generation module 3034 corrects the search tags for which correction instructions were given on the basis of these correction instructions, and stores the corrected content in the search database 3027.

Furthermore, when a medical-related standard glossary is stored in the storage unit 302, the search database data generation module 3034 may search for synonyms on the basis of words included in the medical history information or words obtained by performing natural language processing on the medical history information, or search cosine similarities of the medical history information and the like, obtain words from the standard glossary, and set those words as new search tags in the search database 3027.

Furthermore, the search database data generation module 3034 stores comments about search tags and/or information indicating a deprecated state in the search database 3027 in a state associated with the tags, and when the search database 3027 is searched on the basis of the search tags by the search DB search module 3036, the presentation control unit 3038 presents this comment if a comment is associated with this search tag, and makes the presentation mode of the operation instruction data associated with this tag different from the presentation mode of other operation instruction data if information indicating a deprecated state is associated with the tag.

Furthermore, the search database data generation module 3034 stores flags input for search tags and/or information indicating the number of positive and negative feedbacks in association with these tags in the search database 3027, and when the search database 3027 is searched by the search DB search module 3036, the presentation control unit 3038 filters operation instruction data on the basis of flags or causes the operation instruction data to be displayed preferentially if a flag is associated with a search tag, and causes the operation instruction data to be displayed preferentially if information indicating one of positive feedback and negative feedback is associated with a tag and the number of negative feedbacks is greater than positive feedbacks.

Furthermore, the search database data generation module 3034 stores, in association with operation instruction data in the search database 3027, the number of operation instructions for operation instruction data for each user, the number of operation instructions for operation instruction data for each clinical department to which a user belongs, a name of a patient related to operation instruction data, an age of the patient, a creator of the operation instruction data, a job title of the creator, or a clinical department to which the creator belongs, and when the search database 3027 is searched by the search DB search module 3036, the presentation control unit 3038 changes a display mode of the operation instruction data on the basis of a number of past operation instructions by the user, a number of operation instructions by clinical department, a number of operation instructions at a medical institution, a name of a patient, an age of the patient, a creator of the operation instruction data, a job title of the creator of the operation instruction data, a clinical department to which the creator belongs, a number of operation instructions for operation instruction data based on the operation instruction data in the clinical department to which the user belongs, a ranking of a number of operation instructions based on the operation instruction data for each job title, and a ranking of the number of individual operation instruction data associated with the operation instruction data.

The various information input reception modules 3035 receive various information including medical information input from the terminal apparatuses 10 and 20, the external server 40, and the like, and store the information in the storage unit 302. Preferably, the various information input reception modules 3035 comprise input devices such as a keyboard, a pointing device, and a scanner, and receive various information input via the input devices and store the information in the storage unit 302. Furthermore, the various information input reception modules 3035 receive various information input via the input devices 13 and 23 and the voice processing units 17 and 27 of the terminal apparatuses 10 and 20, and store the information in the storage unit 302.

Furthermore, the various information input reception modules 3035 receive a result of instructing modules in the server 30 and the external server 40 to execute a specific operation on the basis of the operation instruction data by the operation instruction information output module 3039, and update the medical information data 3025 on the basis of this result. The updated medical information becomes input to the search DB search module 3036 and also becomes a source of search tags generated by the search database data generation module 3034. When the medical information is updated by the various information input reception modules 3035, the presentation control unit 3038 may present the updated and corrected content, receive approval of the update via the terminal apparatus 20, and when this approval of the update is received, the various information input reception modules 3035 may update the medical information.

The search DB search module 3036 searches the search database 3027 using natural language included in medical history information or search tags generated from natural language on the basis of various information received by the various information input reception modules 3035, and acquires, as search results, operation instructions for modules in the server 30 and the external server 40 corresponding to the search tags, or medical history information to be input to the natural language generation model 3023 and the operation instruction data generation model 3024. Furthermore, the search by the search DB search module may be performed on the basis of a vector representation based on natural language or a vector representation itself.

The generation model input/output module 3037 inputs various information including medical history information received by the various information input reception modules 3035, and instructions and the like acquired by the search DB search module 3036, to the natural language generation model 3023 and the operation instruction data generation model 3024, and acquires natural language or operation instruction data that is output from the natural language generation model 3023 and the operation instruction data generation model 3024. Furthermore, the generation model input/output module 3037 inputs various information including medical history information received by the various information input reception modules 3035 to the determination model 3029, and acquires a determination result from the determination model 3029. Then, on the basis of the determination result from the determination model 3029, the generation model input/output module 3037 inputs various information including medical information received by the various information input reception modules 3035, and instructions and the like acquired by the search DB search module 3036, to the natural language generation model 3023 and the operation instruction data generation model 3024, and acquires natural language or operation instruction data that is output from the natural language generation model 3023 and the operation instruction data generation model 3024.

The presentation control unit 3038 transmits, in cooperation with the transmission control module 3032, various information including medical history information received by the various information input reception modules 3035, and operation instruction data that is output from the natural language generation model 3023 and the operation instruction data generation model 3024 and acquired by the generation model input/output module 3037, to the terminal apparatus 20 operated by a healthcare professional, and causes the information to be displayed on the display 241 of this terminal apparatus 20. It should be noted that content of text input (including corrections, additions, and deletions) and selection input performed by the healthcare professional operating the terminal apparatus 20 on the basis of various information and the like displayed by the presentation control unit 3038 via the display 241 of the terminal apparatus 20 is acquired by the various information input reception modules 3035 in cooperation with the reception control module 3031.

Furthermore, when outputting instruction information for modules in the server 30 and the external server 40 that is output by the operation instruction information output module 3039 described later, the presentation control unit 3038 causes a user interface that requests approval by a healthcare professional for this instruction information to be displayed on the display 241 of the terminal apparatus 20. As described above, instruction input and selection input performed by the healthcare professional via this interface are acquired by the various information input reception modules 3035 in cooperation with the reception control module 3031. At this time, the presentation control unit 3038 causes information summarizing the operation instruction data that is the source of the instruction information to be displayed via the display 241 of the terminal apparatus 20, and requests approval for the instruction information. This information summarizing the operation instruction data may be generated by the natural language generation model 3023 or the operation instruction data generation model 3034.

Furthermore, when the medical information is a medical history of a specific patient, the presentation control unit 3038 generates and updates a problem list on the basis of this medical history, sets search words on the basis of this problem list, and searches the search database of operation instruction data using these search words. Then, the operation instruction information output module 3039 searches the search database of operation instruction data for machines in the medical institution or for the same patient in the past on the basis of medical history information or tags, obtains operation instruction data such as examination operation instruction candidates, prescription operation instruction candidates, and disease name registration operation instructions for the same patient for whom the medical history is described, and the presentation control unit 3038 causes a user interface that displays tags for these examination operation instruction candidates, prescription operation instruction candidates, and disease name registration operation instruction candidates, presents them to a healthcare professional, and requests approval to be displayed on the display 241 of the terminal apparatus 20. In addition, the frequency of examination instructions and prescription instructions in the past medical information data 3025 is displayed. This frequency display may take various forms, such as changing the display order, changing the color, or changing the density of characters and the like.

The presentation control unit 3038 also has a function of outputting medical history information as input to the determination model 3029, and determining whether to generate, search for, or display operation instruction data as a result of the determination model.

Furthermore, the presentation control unit 3038 presents, via the display 241 of the terminal apparatus 20, at least one of medical history information, information input by a patient, a disease name, a symptom name, a problem in a problem list, and clinical notes that serve as a basis for generation of a search tag, when the search DB search module 3036 searches for operation instruction data based on the tag. At this time, the search database data generation module 3034 records a link with the medical history information and a position of a disease name included in the medical history information in the search database 3027, and when the presentation control unit 3038 presents the medical history information that serves as a basis for generation of a tag, if the medical history information that serves as a basis for generation of the tag is presented for the first time, the presentation control unit 3038 presents that it is presented for the first time, and if there are a plurality of pieces of medical history information that serve as a basis for generation of the tag, the presentation control unit 3038 presents the medical history information that serves as a basis for generation of the tag having the oldest date information.

The operation instruction information output module 3039 generates instruction information for modules in the server 30 and the external server 40 on the basis of natural language that is output from the natural language generation model 3023, operation instruction data that is output from the operation instruction data generation model 3024, and a result of operation input performed by a healthcare professional via the terminal apparatus 20, and outputs the instruction information to these modules.

The EMR data generation module 3040 acquires, as a log, information exchanges between each module of the control unit 303 of the server 30 and the natural language generation model 3023 and the operation instruction data generation model 3024, and stores this log in the storage unit 302 as EMR data 3022.

### 2 Data Structure

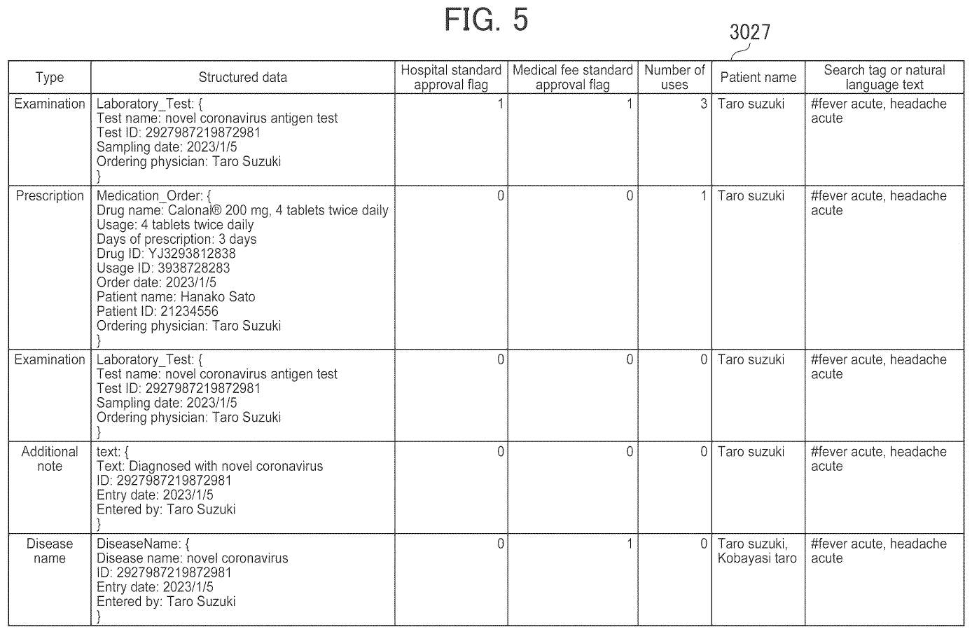

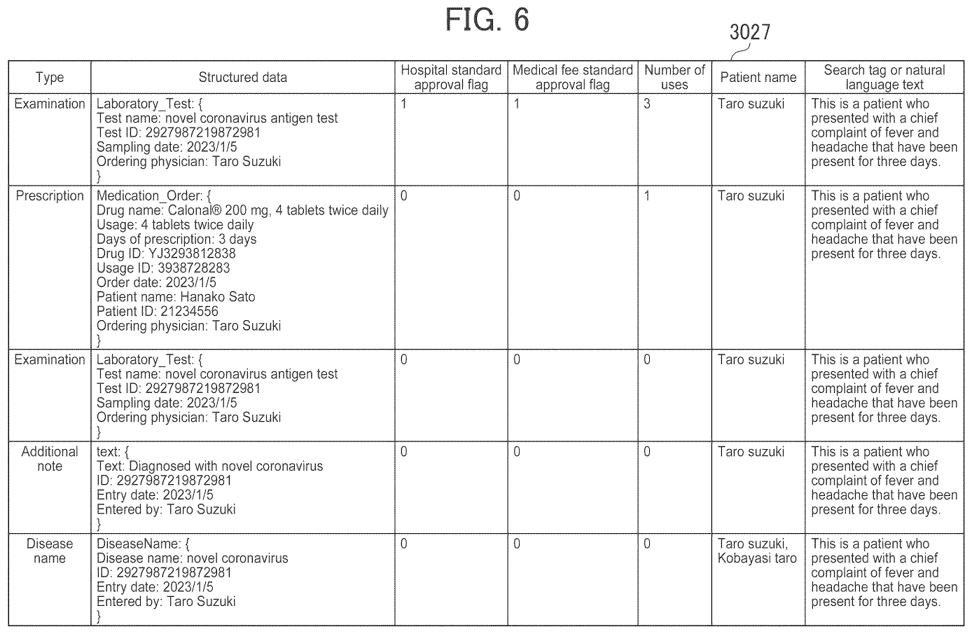

FIGS. 5 and 6 are diagrams showing data structures of databases stored by the server 30. It should be noted that FIGS. 5 and 6 are examples and do not exclude data not described.

The databases shown in FIGS. 5 and 6 refer to relational databases, and are for managing data sets called tables in tabular format that are structurally defined by rows and columns in association with each other. In databases, tables are called tables, table columns are called columns, and table rows are called records. In relational databases, relationships between tables can be set and associated.

Normally, each table is set with a column that serves as a primary key for uniquely specifying a record, but setting a primary key for a column is not mandatory. The control unit 303 of the server 30 can cause the processor 29 to add, delete, and update records in a specific table stored in the storage unit 302 in accordance with various programs.

FIG. 5 is a diagram showing an example of a data structure of the search DB 3027. As shown in FIG. 5, each of the records of the search DB 3027 includes, for example, an item “type”, an item “structured data”, an item “hospital standard approval flag”, an item “clinical department standard approval flag”, an item “number of times used”, an item “patient name”, and an item “search tag or natural sentence”. Each item of the search DB 3027 is input by the search database data generation module 3034. Information stored by the search DB 3027 can be changed and updated as appropriate.

The item “type” is information indicating a type of medical information.

The item “structured data” is structured data associated with the item “search tag or natural sentence”, and is an example of operation instruction data that instructs modules in the server 30 and the external server 40 to execute specific operations.

The item “hospital standard approval flag” and the item “clinical department standard approval flag” are flags set (1 is input to the item) by the operation instruction information output module 3039 when the operation instruction information output module 3039 outputs operation instruction data to modules in the server 30 and the external server 40 to instruct these modules to execute specific operations, the presentation control unit 3038 causes buttons and the like for inputting whether or not to approve this operation instruction data as a hospital standard and whether or not to approve it as a clinical department standard to be displayed on the display 241 of the terminal apparatus 20, and an input indicating approval is accepted.

The item “number of times used” is incremented by the operation instruction information output module 3039 each time the operation instruction information output module 3039 outputs operation instruction data specified by the item “structured data” to modules in the server 30 and the external server 40.

The item “patient name” is information relating to a patient's name that was included in medical information that is a source for generating data of the relevant row of the search DB 3027 when generating the data.

“Search tag or natural sentence” is information indicating a search tag included in medical information specified by the item “type”.

FIG. 6 is a diagram showing another example of a data structure of the search DB 3027. As shown in FIG. 6, each of the records of the search DB 3027 includes, for example, an item “type”, an item “structured data”, an item “hospital standard approval flag”, an item “clinical department standard approval flag”, an item “number of times used”, an item “patient name”, and an item “search tag or natural sentence”. Each item of the search DB 3027 is input by the search database data generation module 3034. Information stored by the search DB 3027 can be changed and updated as appropriate.

Since the data structure shown in FIG. 6 and the data structure shown in FIG. 5 are substantially the same, detailed description is omitted, but in FIG. 6, a natural sentence is input to the item “search tag or natural sentence”.

### 3 Operation Example

Hereinafter, an example of an operation of the server 30 will be described.

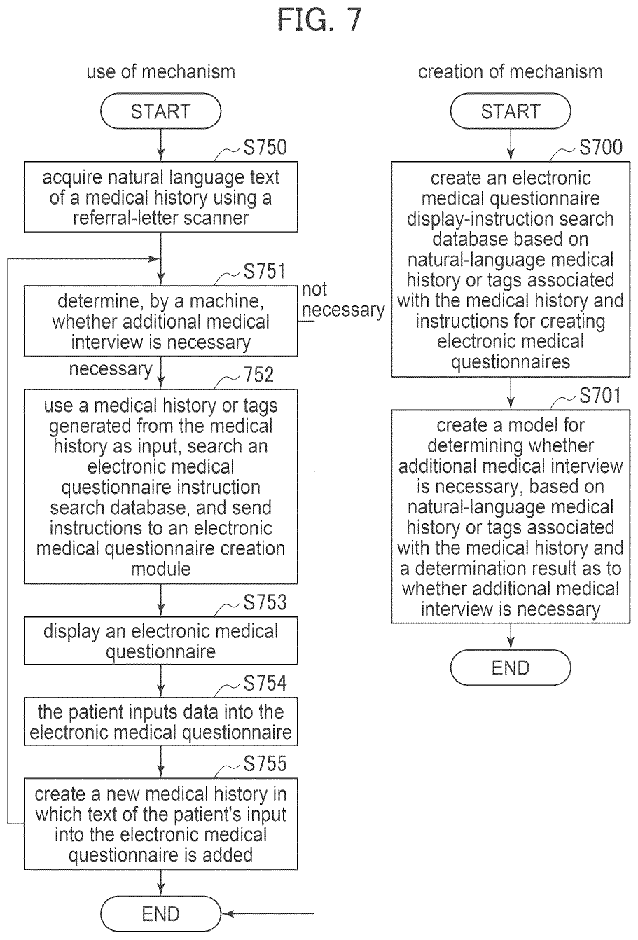

FIG. 7 is a flowchart illustrating an example of an operation of the server 30. FIG. 7 is a diagram illustrating an example of an operation in which, starting from a referral letter from another hospital brought by a patient, operation instruction data for an electronic medical interview form is generated or extracted from a search database on the basis of medical history information described in the referral letter, and when there is a response from the patient to the electronic medical interview form, the response is added to the medical history information.

The flowchart shown on the right side of FIG. 7 illustrates an operation for generating the search database 3027 and the determination model 3029 for realizing the operation shown in the flowchart on the left side of FIG. 7, and the flowchart shown on the left side of FIG. 7 illustrates an operation based on the search database 3027 and the determination model 3029 generated by the operation shown in the flowchart on the right side of FIG. 7.

First, the flowchart shown on the right side of FIG. 7 will be described. In step S700, the control unit 303 refers to the electronic medical record (EMR) data 3022 and the like, extracts medical history information described in natural language or search tags linked to the medical history and an electronic medical interview form display execution instruction from a healthcare professional included in the EMR data 3022, and creates an electronic medical interview form display instruction search database, which is an example of the search database 3027. Specifically, for example, the control unit 303, by means of the search database data generation module 3034, refers to the EMR data 3022 and the like, extracts natural language of the medical history or search tags linked to the medical history and an electronic medical interview form creation instruction, and creates an electronic medical interview form display instruction search database, which is an example of the search database 3027. The electronic medical interview form display instruction search database generated in step S700 is stored in the search database 3027 of the storage unit 302.

Next, in step S701, the control unit 303 refers to the EMR data 3022 and the like, extracts natural language of the medical history or search tags linked to the medical history and a determination result as to whether an additional medical interview by a healthcare professional is necessary, included in the medical information, and creates a determination model for determining whether an additional medical interview is necessary, which is an example of the determination model 3029. Specifically, for example, the control unit 303, by means of the generation model generation module 3033, refers to the EMR data 3022 and the like, extracts natural language of the medical history or search tags linked to the medical history and a determination result as to whether an additional medical interview by a healthcare professional is necessary, included in the medical information, and creates a determination model for determining whether an additional medical interview is necessary, which is an example of the determination model 3029. The determination model for determining whether an additional medical interview is necessary, generated in step S701, is stored in the determination model 3029 of the storage unit 302.

Next, the flowchart shown on the left side of FIG. 7 will be described. In step S750, the control unit 303 obtains medical history information, particularly natural language of the medical history, by scanning a referral letter from another hospital brought by the patient. Specifically, for example, the control unit 303 scans the referral letter by means of a scanner provided in the various information input reception module 3035, and obtains medical history information, particularly natural language of the medical history, from the referral letter. Then, the various information input reception module 3035 stores the obtained natural language of the medical history in the storage unit 302 as medical information data 3025.

In step S751, the control unit 303 inputs the natural language of the medical history obtained in step S750 into the determination model 3029, and obtains a determination result. The determination result of the determination model 3029 in FIG. 7 is a determination result as to whether an additional medical interview is necessary for the patient, based on the natural language of the medical history obtained in step S750. Specifically, for example, the control unit 303, by means of the generation model input/output module 3037, inputs the natural language of the medical history obtained in step S750 into the determination model 3029, and obtains a determination result. Then, if the determination result by the determination model 3029 is a determination result that an additional medical interview is necessary for the patient, the program proceeds to step S752, and if it is determined that it is not included, the operation according to the flowchart on the left side of FIG. 7 is terminated.

In step S752, the control unit 303 searches an electronic medical interview form instruction search database, which is an example of the search database 3027, using the medical history or a search tag generated from the medical history as a search key, transmits operation instruction data, which is a search result, to an electronic medical interview form generation module, which is an example of a module in the server 30 or the external server 40, and instructs creation of electronic medical interview form data using the electronic medical interview form generation module. Specifically, for example, the control unit 303, by means of the search DB search module 3036, searches an electronic medical interview form instruction search database, which is an example of the search database 3027, using the medical history or a search tag generated from the medical history as a search key, and by means of the execution instruction information output module 3039, transmits operation instruction data, which is a search result, to an electronic medical interview form generation module, which is an example of a module in the server 30 or the external server 40, and instructs creation of electronic medical interview form data using the electronic medical interview form generation module.

In step S753, the control unit 190 of the terminal apparatus 10 possessed by the patient causes the display 141 to display an electronic medical interview form on the basis of electronic medical interview form data transmitted from the electronic medical interview form creation module of the server 30. Next, in step S754, the patient inputs data into the electronic medical interview form using the terminal apparatus 10. The input data is transmitted to the server 30 via the control unit 190 of the terminal apparatus 10.

In step S755, the control unit 303 adds the electronic medical interview form data input and transmitted in step S754 to the medical history information, and generates new (updated) medical history information. Specifically, for example, the control unit 303, by means of the various information input reception module 3035, adds the electronic medical interview form data input and transmitted in step S754 to the medical history information, and generates new (updated) medical history information. Note that a medication record scanner may be used instead of the referral letter scanner described above. Also, as an example, in guiding medical treatment, when the onset of a COVID-19 infection is anticipated and the prescription of a drug called Paxlovid, which has many interacting drugs, is being considered, the system may have a function of highlighting and displaying drugs that interact with Paxlovid when displaying the OCR result data of the medication record, and displaying whether the patient is at high risk for prescribing such drugs.

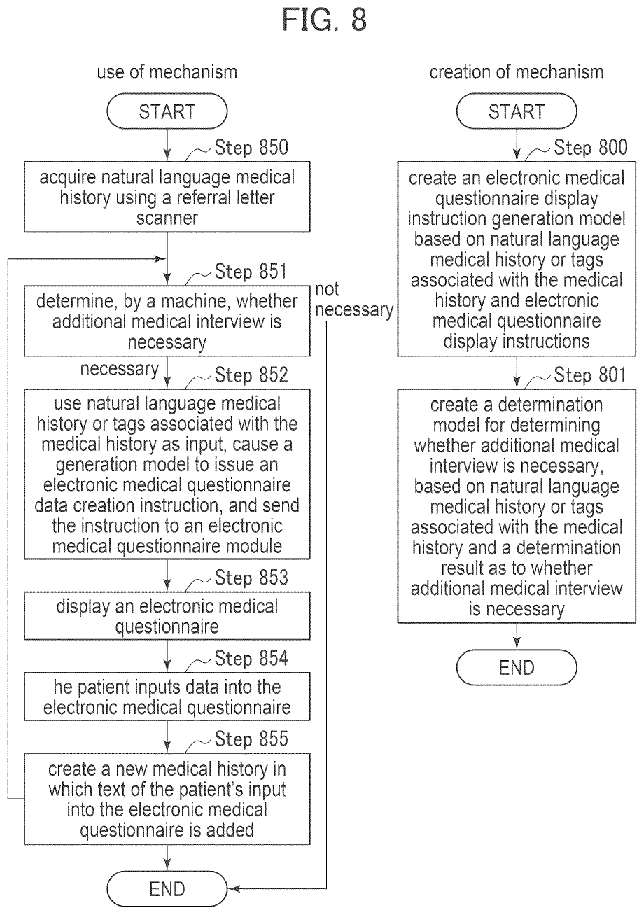

FIG. 8 is a flowchart illustrating an example of an operation of the server 30. The operation shown in the flowchart of FIG. 8 is similar to the operation shown in the flowchart of FIG. 7, but differs in that while FIG. 7 generates data of the search database 3027, FIG. 8 generates the operation instruction data generation model 3024. Accordingly, the description of steps common to the description of FIG. 7 will be omitted, and the description will focus on steps different from those of FIG. 7.

In step S800 of the flowchart shown on the right side of FIG. 8, the control unit 303 refers to the electronic medical record (EMR) data 3022 and the like to extract natural language of medical history information or search tags associated with medical history information, and an electronic medical interview form creation instruction by a healthcare professional included in the medical information, and creates an electronic medical interview form display instruction generation model, which is an example of the operation instruction data generation model 3024. Specifically, for example, the control unit 303 uses the generation model generation module 3033 to refer to the EMR data 3022 and the like to extract natural language of medical history information or search tags associated with medical history information and an electronic medical interview form creation instruction, and creates an electronic medical interview form display instruction generation model, which is an example of the operation instruction data generation model 3024. The electronic medical interview form display instruction generation model generated in step S800 is stored in the operation instruction data generation model 3024 of the storage unit 302.

In step S852 of the flowchart shown on the left side of FIG. 8, the control unit 303 inputs medical history information or tags generated from medical history information into the electronic medical interview form display instruction generation model, which is an example of the operation instruction data generation model 3024, and uses the operation instruction data that is the output result of the electronic medical interview form display instruction generation model to instruct the electronic medical interview form generation module, which is an example of a module in the server 30 or the external server 40, to create electronic medical interview form data. Specifically, for example, the control unit 303 uses the generation model input/output module 3037 to input medical history information or tags generated from medical history information into the electronic medical interview form display instruction generation model, which is an example of the operation instruction data generation model 3024, and uses the operation instruction information output module 3039 to instruct the electronic medical interview form generation module, which is an example of a module in the server 30 or the external server 40, to create electronic medical interview form data using the operation instruction data that is the output result of the electronic medical interview form display instruction generation model.

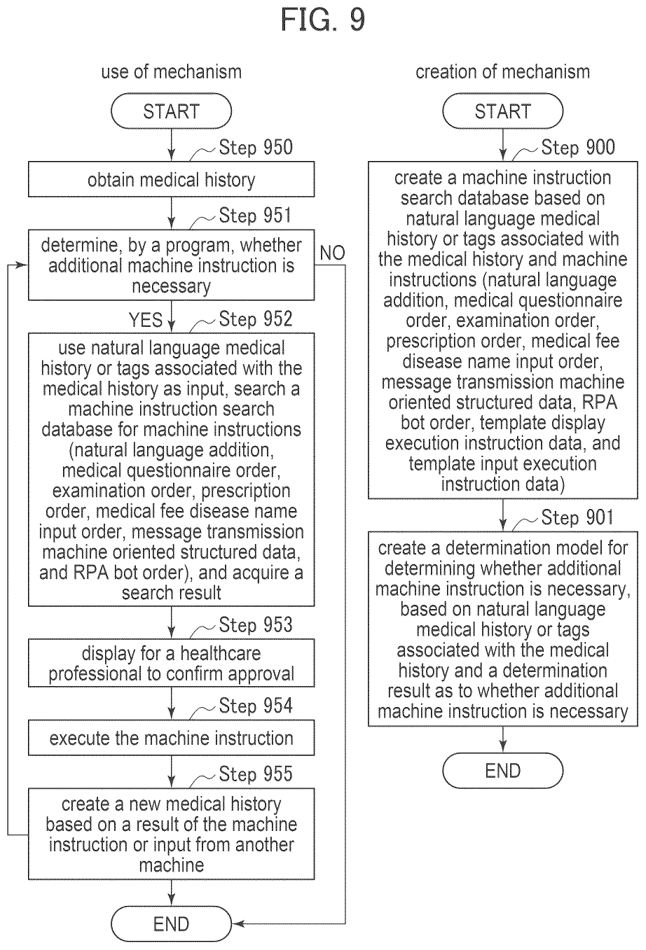

FIG. 9 is a flowchart showing an example of the operation of the server 30. FIG. 9 is a diagram showing an example of an operation in which medical history information, which is an example of medical information included in the EMR data 3022 and the like, is used as a starting point, operation instructions are given to modules in the server 30 or the external server 40 based on this medical history information, and the medical history information is updated based on the processing results from these modules.

The flowchart shown on the right side of FIG. 9 shows an operation for generating the search database 3027 and the determination model 3029 to implement the operation shown in the flowchart on the left side of FIG. 9, and the flowchart shown on the left side of FIG. 9 shows an operation based on the search database 3027 and the determination model 3029 generated by the operation shown in the flowchart on the right side of FIG. 9.

First, the flowchart shown on the right side of FIG. 9 will be described. In step S900, the control unit 303 refers to the EMR data 3022 and the like to create a machine instruction search database, which is an example of the search database 3027, from medical history information described in natural language or search tags associated with medical history information, and operation instruction data related to the medical history information in the EMR data 3022 (electronic medical interview form generation operation instruction data, examination order operation instruction data, prescription order operation instruction data, disease name registration operation instruction data, message transmission operation instruction data, RPA operation instruction data, or natural language addition operation instruction data). That is, the control unit 303 uses the search database data generation module 3034 to refer to the EMR data 3022 and the like, and creates a machine instruction search database using, as data, pairs of natural language medical history information or search tags associated with medical history information, and operation instruction data in the EMR data 3022 (electronic medical interview form generation operation instruction data, examination order operation instruction data, prescription order operation instruction data, disease name registration operation instruction data, message transmission operation instruction data, RPA operation instruction data, or natural language addition operation instruction data) that are issued to the same patient as the medical history information at the same time (same year, same month, same hour, same second, etc.) or intentionally associated by a healthcare professional. The machine instruction search database generated in step S900 is stored in the search database 3027 of the storage unit 302.

Next, in step S901, the control unit 303 refers to the EMR data 3022 and the like to create a determination model that takes as input natural language of medical history information or search tags associated with medical history information and determines whether additional machine instructions are necessary. Specifically, for example, the control unit 303 refers to the EMR data 3022 and the like to extract natural language of medical history information or search tags associated with medical history information, and results of whether additional machine instructions were given that are included in the EMR data 3022 and the like, or collects results of healthcare professionals' determinations of whether additional machine instructions are necessary, and creates a determination model by performing machine learning on that data. The determination model for determining whether additional machine instructions are necessary, generated in step S901, is stored in the determination model 3029 of the storage unit 302.

Next, the flowchart shown on the left side of FIG. 9 will be described. In step S950, the control unit 303 obtains medical history information from medical information such as the EMR data 3022. Specifically, for example, the control unit 303 uses the various information input acceptance module 3035 to obtain medical history information from medical information such as the EMR data 3022.

In step S951, the control unit 303 inputs the medical history information obtained in step S950 into the determination model 3029 and obtains a determination result. The determination result of the determination model 3029 in FIG. 9 is a determination result of whether additional machine instructions are necessary based on the medical history information obtained in step S950. Specifically, for example, the control unit 303 uses the generation model input/output module 3037 to input the medical history information obtained in step S950 into the determination model 3029 and obtains a determination result. Then, if the determination result by the determination model 3029 is a determination result that additional machine instructions are necessary, the program proceeds to step S952, and if it is determined that they are not included, the operation according to the flowchart on the left side of FIG. 9 is terminated.

In step S952, the control unit 303 searches the machine instruction search database, which is an example of the search database 3027, using natural language of medical history information or search tags generated from medical history information as a search key, and obtains operation instruction data that is the search result. Specifically, for example, the control unit 303 uses the search database search module 3036 to search the machine instruction search database, which is an example of the search database 3027, using natural language of medical history information or search tags generated from medical history information as a search key, and obtains operation instruction data that is the search result.

In step S953, the control unit 303 transmits the operation instruction data obtained in step S952 to the terminal apparatus 20 possessed by the healthcare professional, and the presentation control unit 294 of the control unit 290 of the terminal apparatus 20 presents this operation instruction data on the display 241 based on the operation instruction data transmitted from the server 30, and further, the instruction information input instruction unit 295 of the terminal apparatus 20 requests the healthcare professional to provide an approval input as to whether to instruct modules in the server 30 or the external server 40 to perform a specific operation based on this operation instruction data. Then, when an approval input is made in step S953, in step S954, the control unit 290 of the terminal apparatus 20 transmits to the server 30 that the approval input has been made, and the control unit 303 transmits the operation instruction data for which the approval input has been made to modules in the server 30 or the external server 40, and instructs a specific operation based on this operation instruction data. Specifically, for example, the control unit 303 uses the operation instruction information output module 3039 to transmit the operation instruction data for which the approval input has been made to modules in the server 30 or the external server 40, and instructs a specific operation based on this operation instruction data.

In step S955, the control unit 303 accepts the result of a specific operation based on the operation instruction data transmitted to modules in the server 30 or the external server 40 in step S954, and updates the medical history information based on this result and input from the modules in the server 30 or the external server 40. Specifically, for example, the control unit 303 uses the various information input acceptance module 3035 to accept the result of a specific operation based on the operation instruction data transmitted to modules in the server 30 or the external server 40 in step S954, and updates the medical history information based on this result and input from the modules in the server 30 or the external server 40.

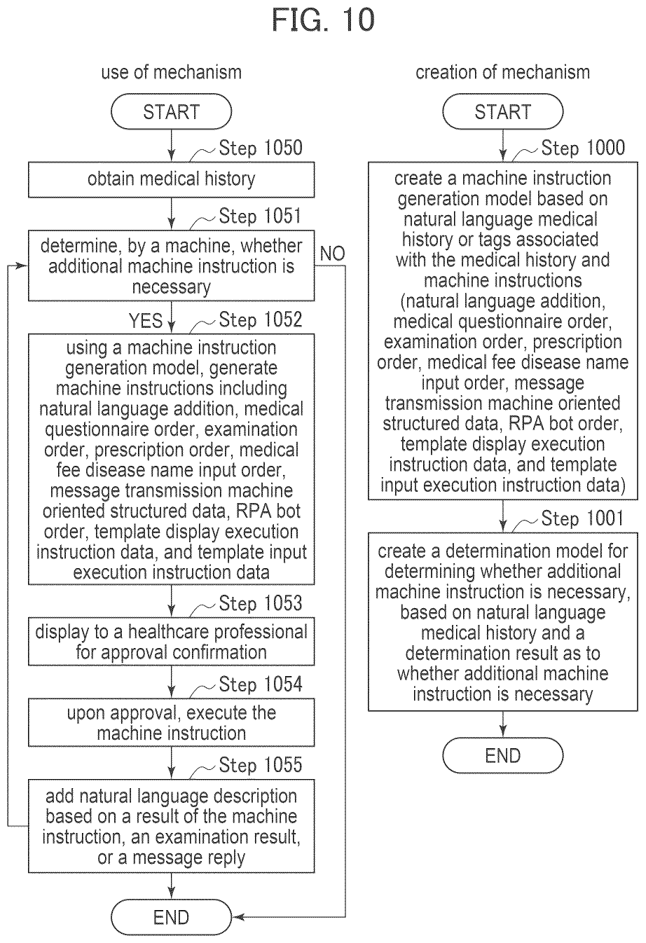

FIG. 10 is a flowchart showing an example of the operation of the server 30. The operation shown in the flowchart of FIG. 10 is similar to the operation shown in the flowchart of FIG. 9, but differs in that while FIG. 9 generates the search database 3027, FIG. 10 generates the operation instruction data generation model 3024. Therefore, descriptions of steps common to the description of FIG. 9 are omitted, and the description focuses on steps different from those in FIG. 7.

In step S1000 of the flowchart shown on the right side of FIG. 10, the control unit 303 refers to the EMR data 3022 and the like to create a machine instruction generation model, which is an example of the operation instruction data generation model 3024, from natural language of medical history information or search tags associated with medical history information, and instructions for modules in the server 30 or the external server 40 (electronic medical interview form generation operation instruction data, examination order operation instruction data, prescription order operation instruction data, disease name registration operation instruction data, message transmission operation instruction data, RPA operation instruction data, or natural language addition operation instruction data). That is, the control unit 303 uses the generation model generation module 3033 to refer to the EMR data 3022 and the like, and creates a machine instruction generation model, which is an example of the operation instruction data generation model 3024, from natural language of medical history information or search tags associated with medical history information, and instructions for modules in the server 30 or the external server 40 (electronic medical interview form generation operation instruction data, examination order operation instruction data, prescription order operation instruction data, disease name registration operation instruction data, message transmission operation instruction data, RPA operation instruction data, or natural language addition operation instruction data). The machine instruction generation model generated in step S1000 is stored in the operation instruction data generation model 3024 of the storage unit 302.

In step S1052 of the flowchart shown on the left side of FIG. 10, the control unit 303 inputs medical history information or tags generated from medical history information into the machine instruction generation model, which is an example of the operation instruction data generation model 3024, and uses the operation instruction data that is the output result of the machine instruction generation model to instruct the electronic medical interview form generation module, which is an example of a module in the server 30 or the external server 40, to create electronic medical interview form data. Specifically, for example, the control unit 303 uses the generation model input/output module 3037 to input medical history information or tags generated from medical history information into the machine display instruction generation model, which is an example of the operation instruction data generation model 3024, and uses the operation instruction information output module 3039 to instruct the electronic medical interview form generation module, which is an example of a module in the server 30 or the external server 40, to create electronic medical interview form data using the operation instruction data that is the output result of the electronic medical interview form display instruction generation model.

### 4. Screen Examples

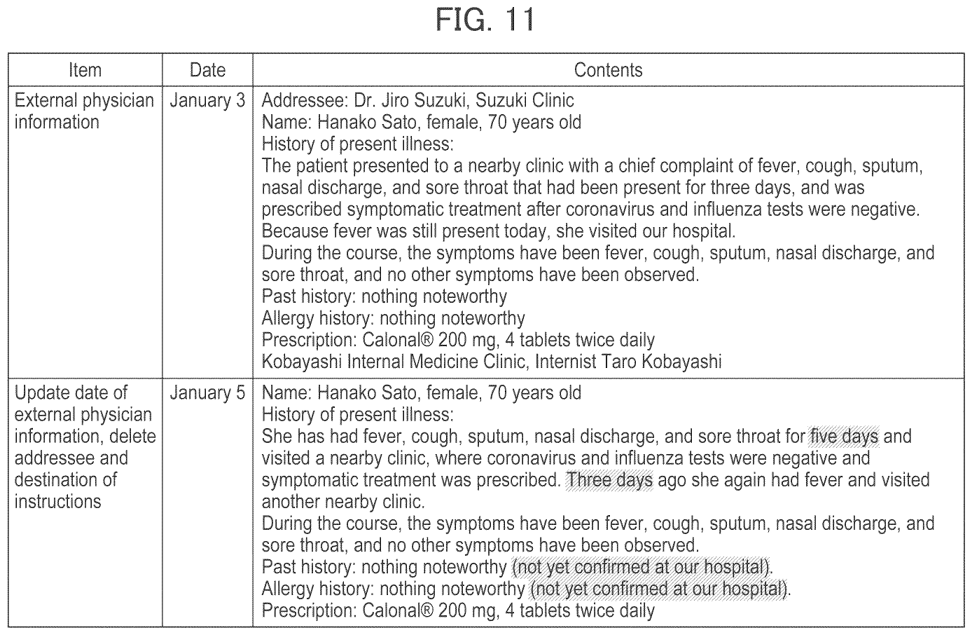

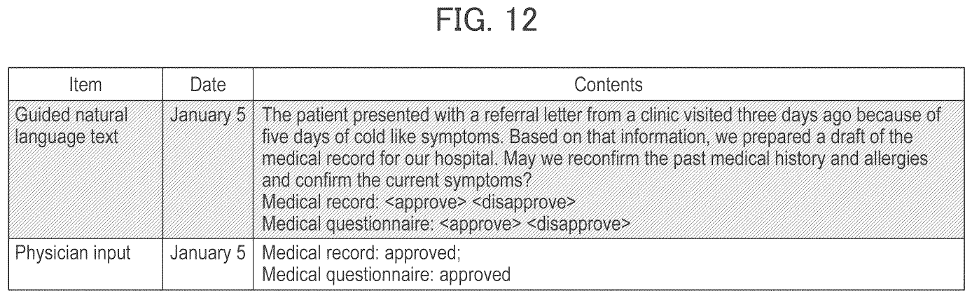

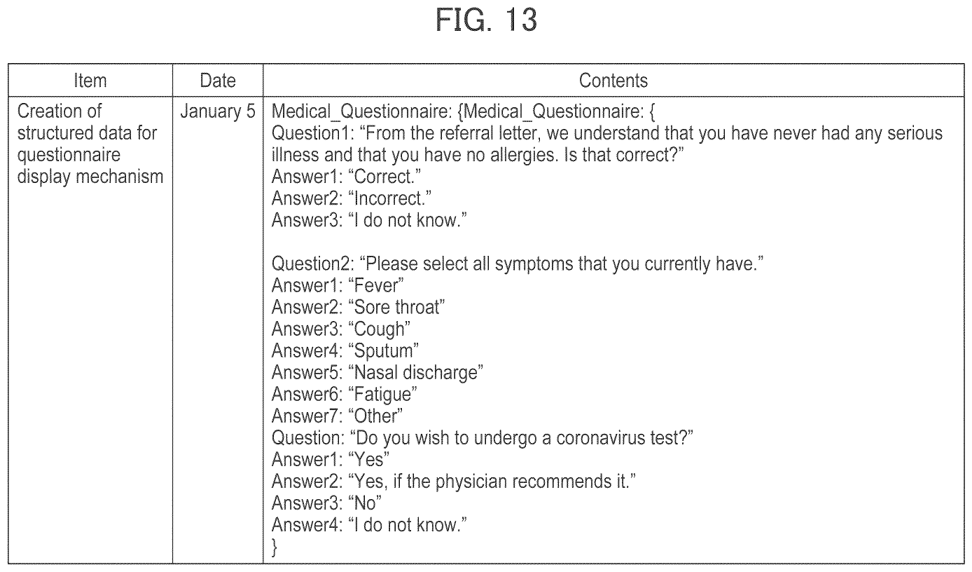

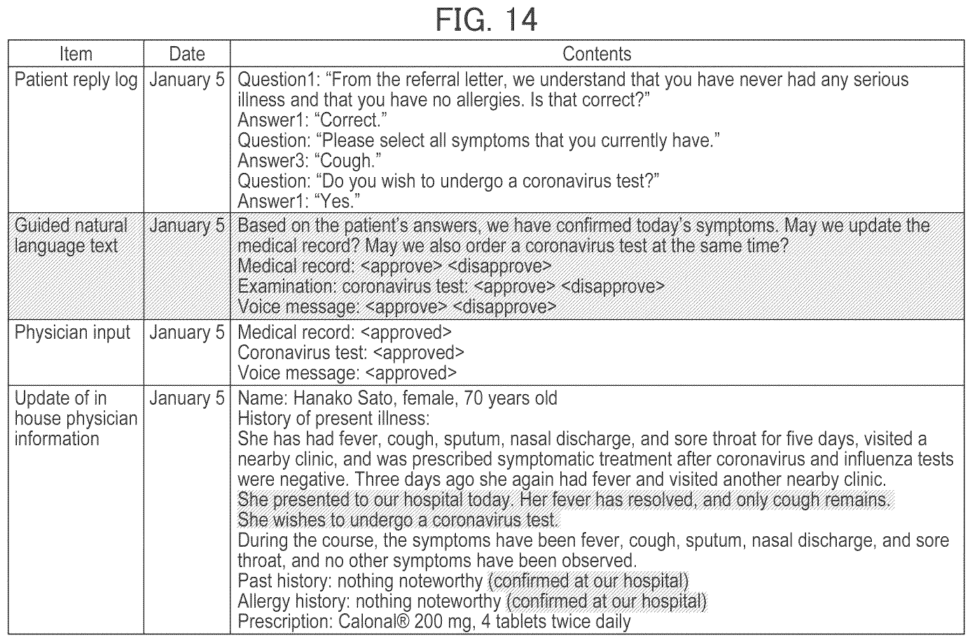

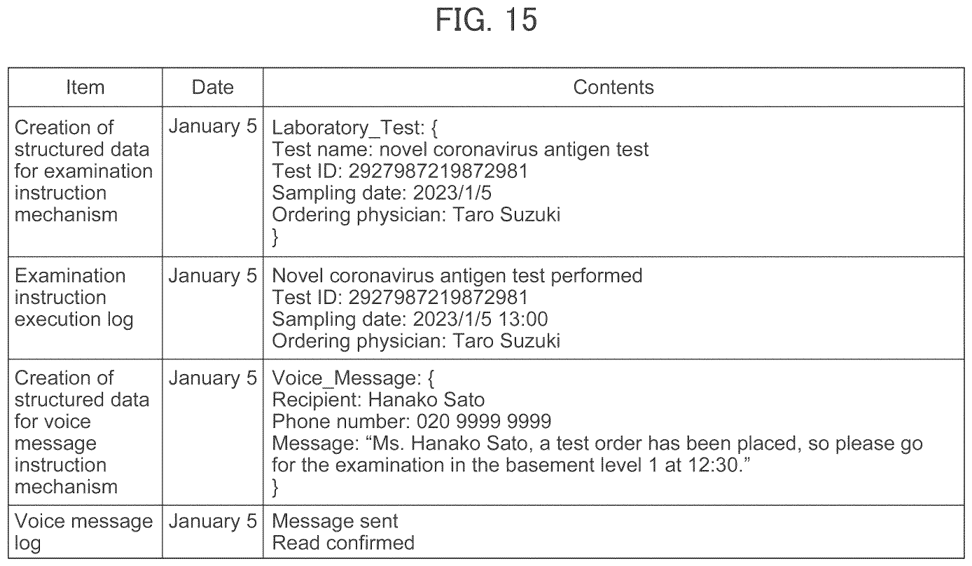

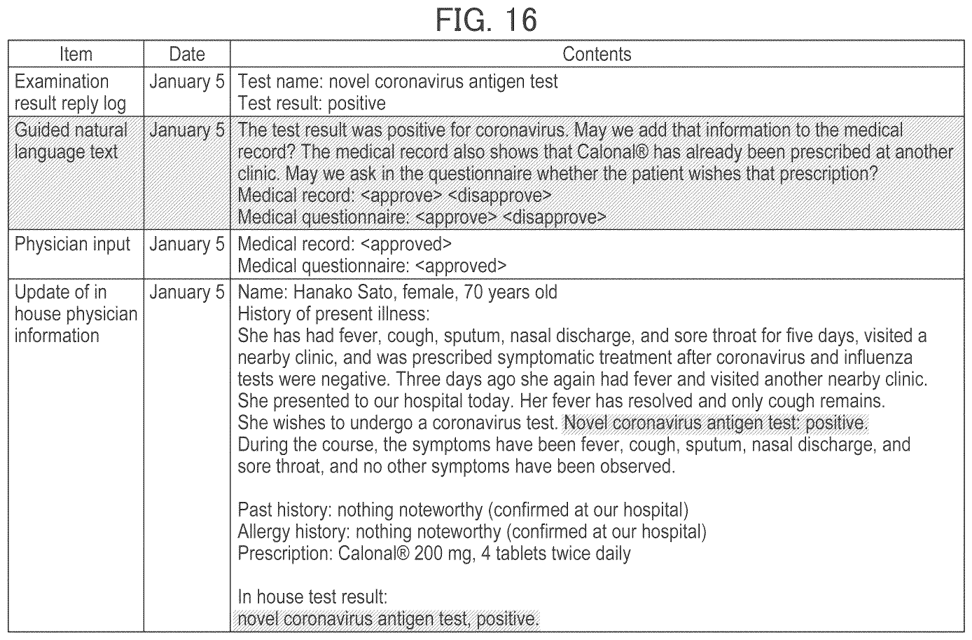

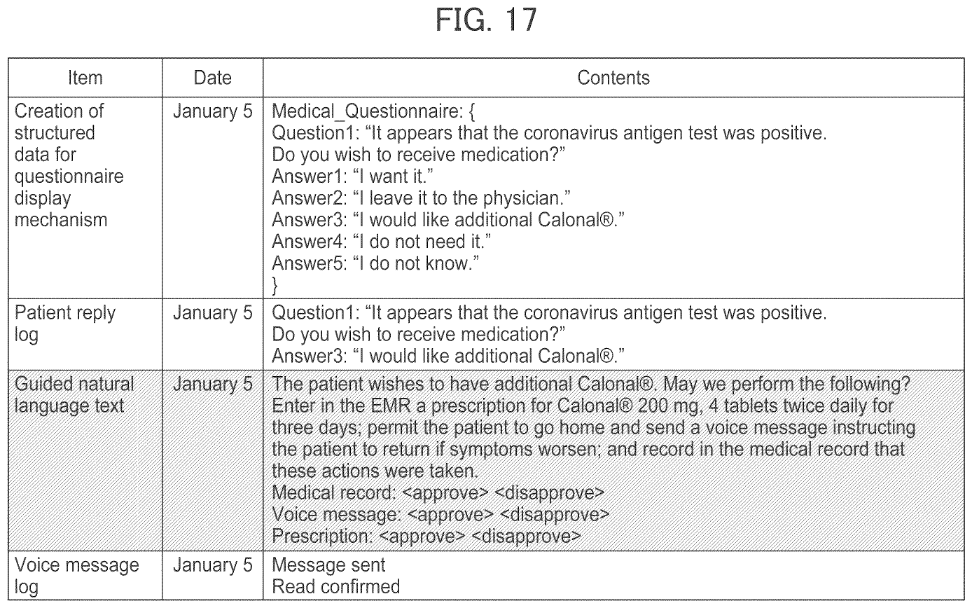

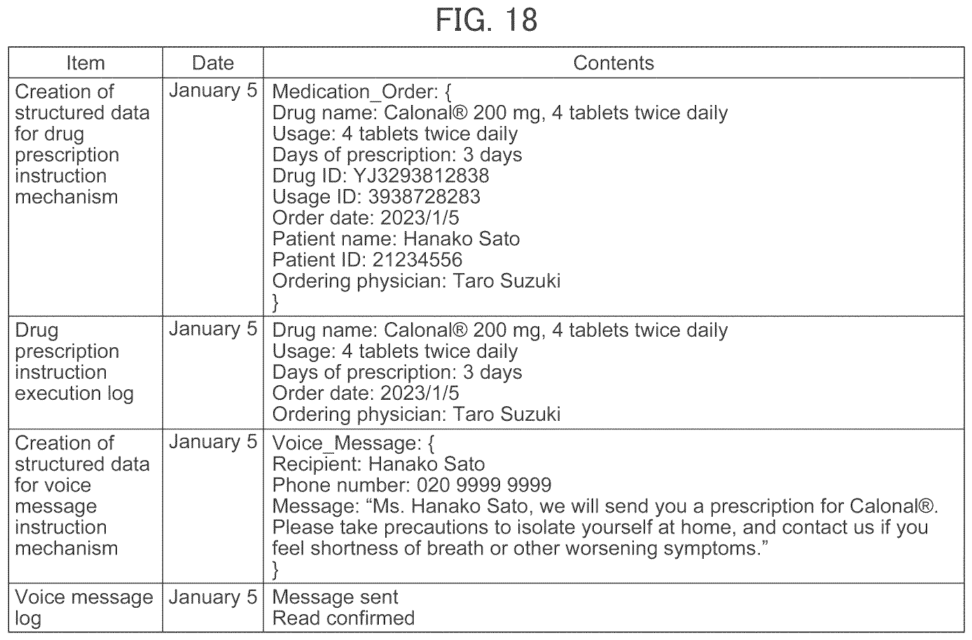

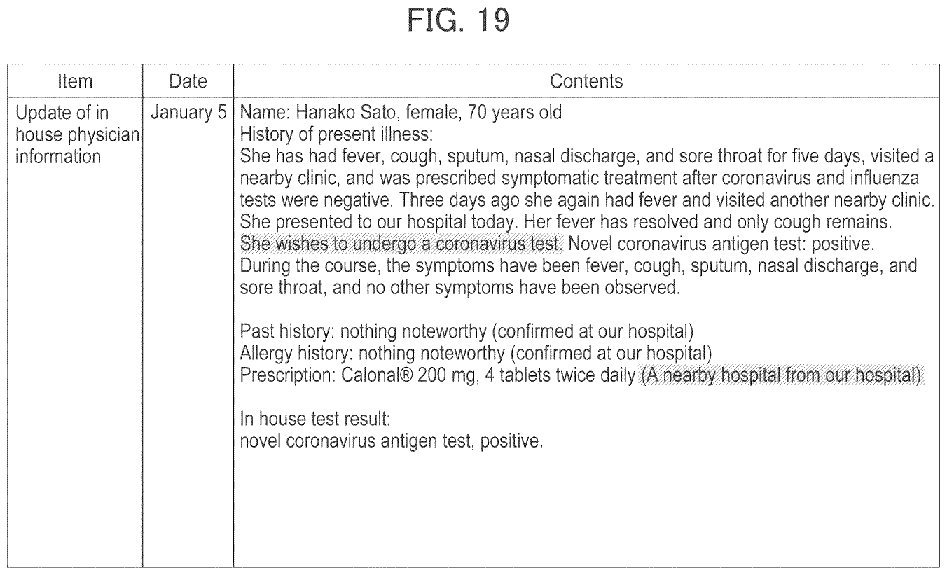

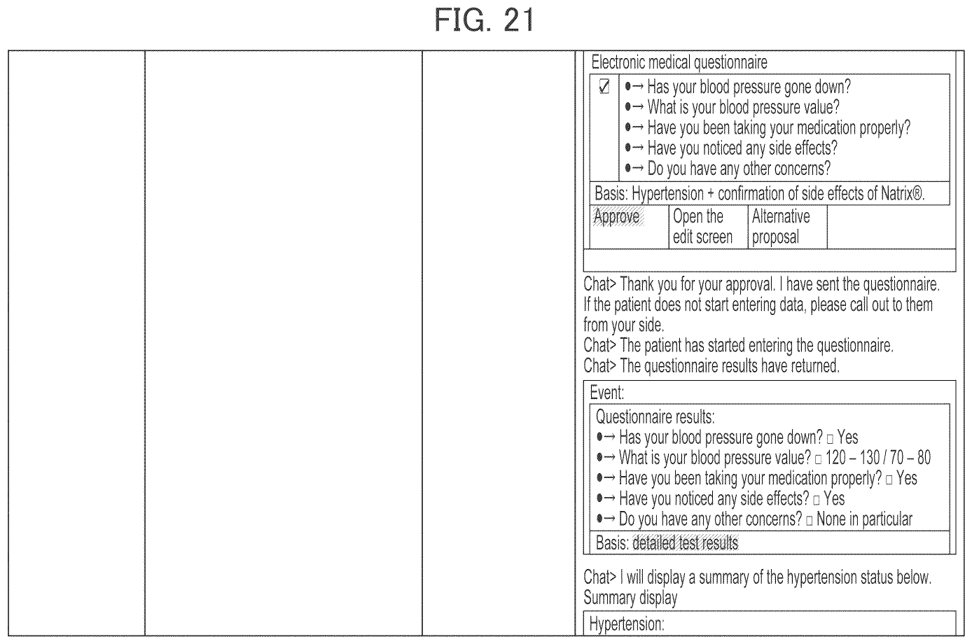

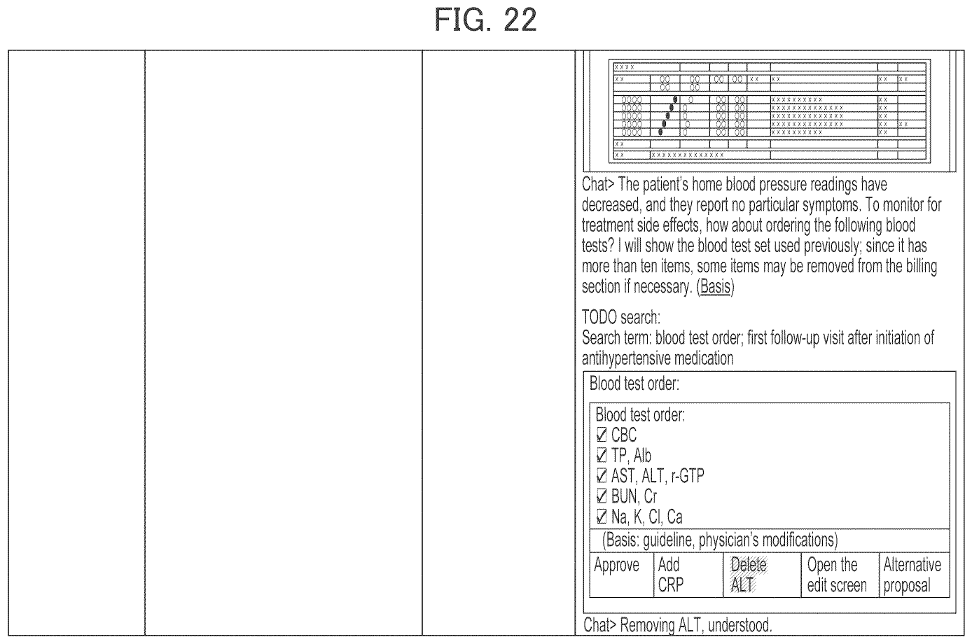

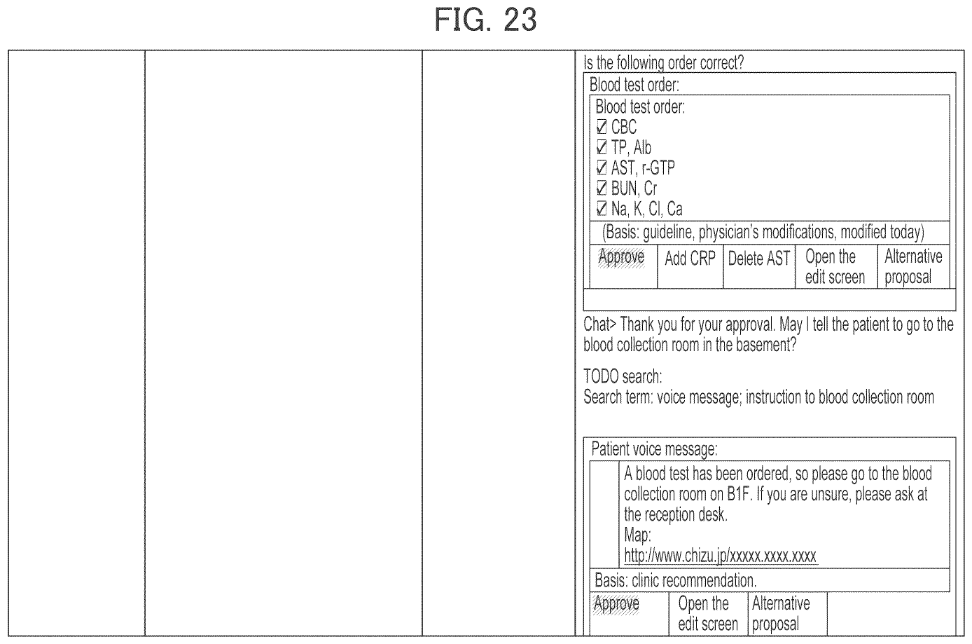

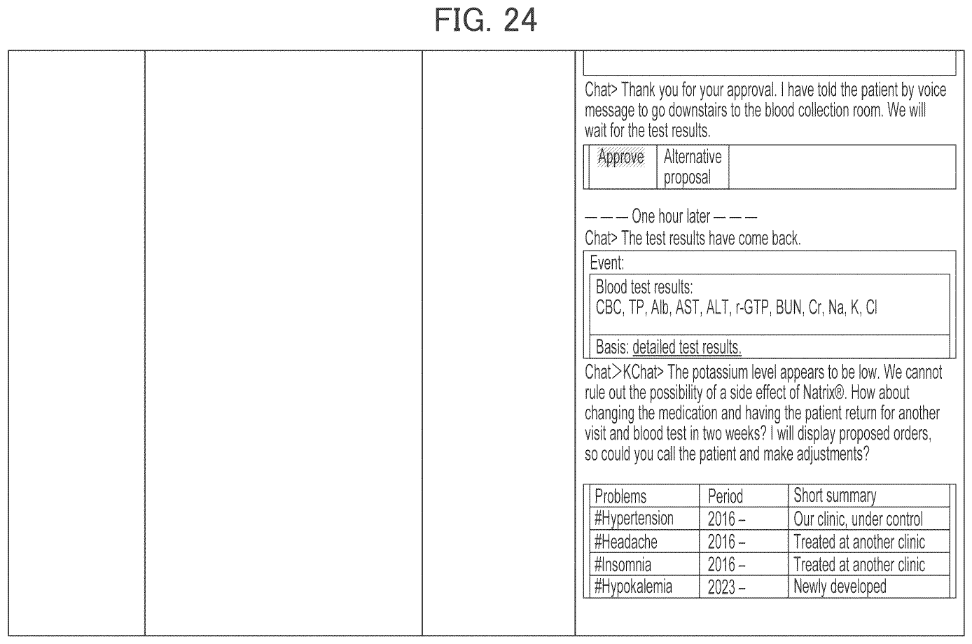

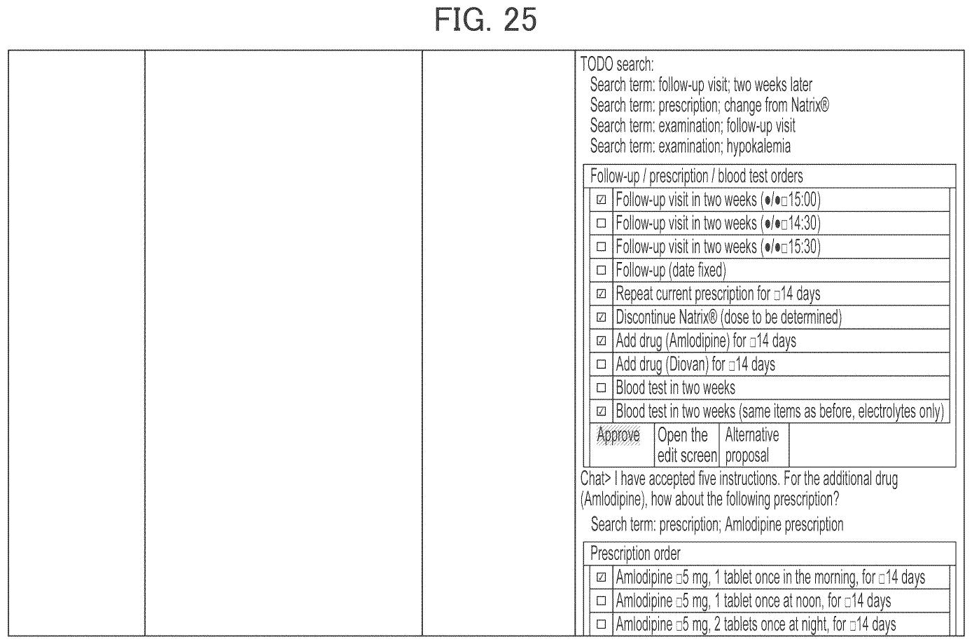

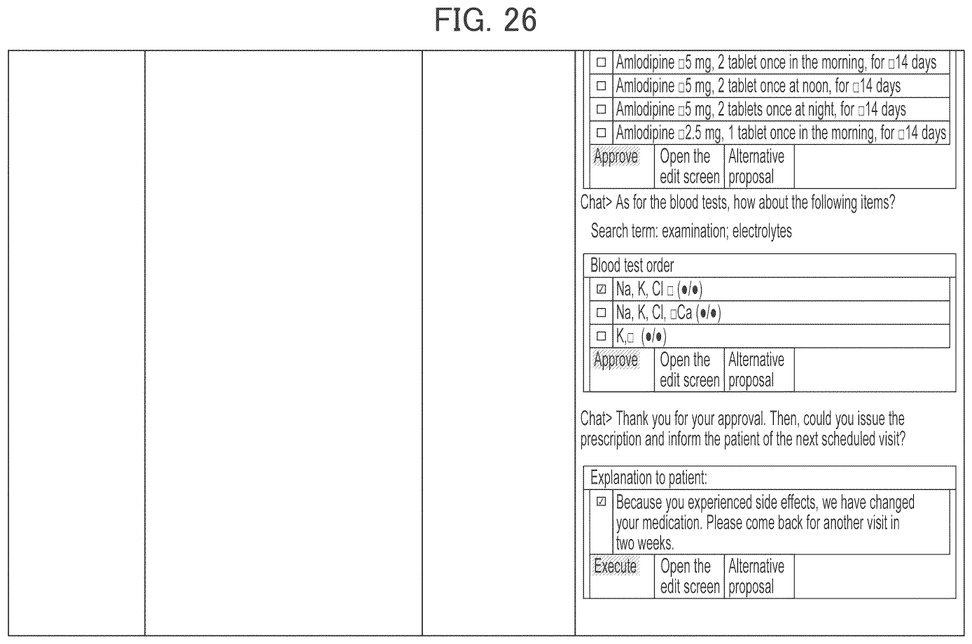

Hereinafter, examples of screens output to the terminal apparatus 20 will be described with reference to FIGS. 11 to 28.

FIGS. 10 to 19 are diagrams showing updates of medical history information in the system 1 of the present embodiment. In these figures, “Item” is information indicating what operation was performed in the system 1, “Date” is information indicating the date on which the operation was performed, and “Content” is medical history information, information exchange in natural language with the natural language generation model 3023 and the operation instruction data generation model 3024, and information indicating instruction information given to modules in the server 30 or the external server 40 and results thereof. The information described in Item, Date, and Content is operation instruction data, and at least part of it becomes a log for generating EMR data.

An outline of the flow of updating medical history information shown in FIGS. 10 to 19 will be described. Since a patient who brought a referral letter from another medical institution came for a consultation, medical history information is obtained by scanning this referral letter. After deleting items and content that do not need to be described in the EMR from the obtained medical history information, when this medical history information is input into the natural language generation model 3023 and the operation instruction data generation model 3024, the natural language generation model 3023 and the operation instruction data generation model 3024 create a draft of a clinical note, create operation instruction data for creating an electronic medical interview form for the patient, and request permission from the physician.

Once permission is obtained from the physician, the clinical note is updated, and this operation instruction data is output to modules in the server 30 or the external server 40 to generate an electronic medical interview form and display it on the patient's terminal apparatus 10. The electronic medical interview form data input by the patient using the terminal apparatus 10 is transmitted to the server 30, and the clinical note is updated with the physician's permission.

Furthermore, the natural language generation model 3023 and the operation instruction data generation model 3024 recommend a COVID-19 test for the patient, and an examination instruction is transmitted to modules in the server 30 or the external server 40 under the physician's instruction. Along with this, a voice instruction instructing the patient to go to the examination room is transmitted to modules in the server 30 or the external server 40.

Furthermore, when the examination result is transmitted to the server 30, based on this examination result, updating the clinical note and creating an additional electronic medical interview form are recommended to the physician, and the clinical note is updated and the electronic medical interview form is transmitted with the physician's permission.

When the patient inputs the electronic medical interview form and inputs a request for additional prescription drugs, the natural language generation model 3023 and the operation instruction data generation model 3024 request the physician to update the clinical note, transmit a voice message to the patient, and determine whether to add prescription drugs, and with the physician's permission, a prescription is issued and a voice message permitting the patient to go home is transmitted to the patient.

In FIGS. 10 to 19, the shaded portions indicate content input by the physician using the terminal apparatus 20.

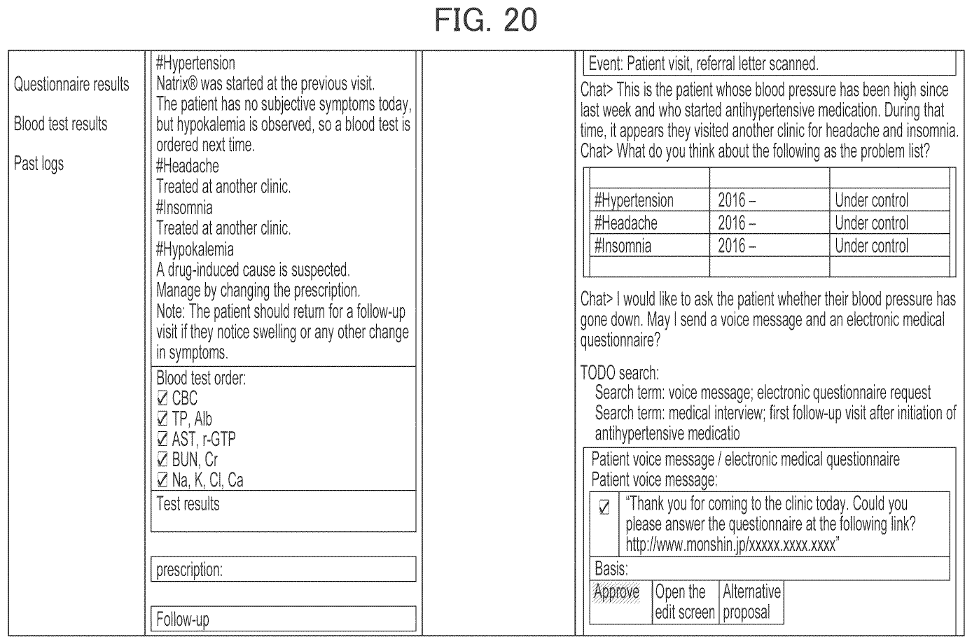

Next, FIGS. 20 to 26 are diagrams showing examples in which outputs from the system 1 of the present embodiment, particularly the server 30 (including the natural language generation model 3023 and the operation instruction data generation model 3024), and input content by a healthcare professional are displayed on the display 241 of the terminal apparatus 20 possessed by the healthcare professional.

The feature of the screen examples shown in FIGS. 20 to 26 is that an input/output log of information between the server 30 and the terminal apparatus 20, and a summary of logs extracted from this input/output log (this summary also becomes part of the EMR data) are displayed on the same screen of the display 241.

On the right side of the screen of the display 241, natural language output by the chat function of the natural language generation model 3023 and the operation instruction data generation model 3024, and instruction input candidates for modules in the server 30 or the external server 40 that are recommended to the healthcare professional and similarly output by the natural language generation model 3023 and the operation instruction data generation model 3024, are displayed. On the other hand, on the left side of the screen of the display 241, a summary of the content displayed on the right side of the screen is generated by the server 30 and displayed. This summary is the clinical note for the healthcare professional and is also the EMR data. The summary is generated by the natural language generation model 3023, the operation instruction data generation model 3024, or a machine learning model trained for summary creation.

In FIGS. 20 to 26, the shaded portions indicate options selected and input by the physician using the terminal apparatus 20.

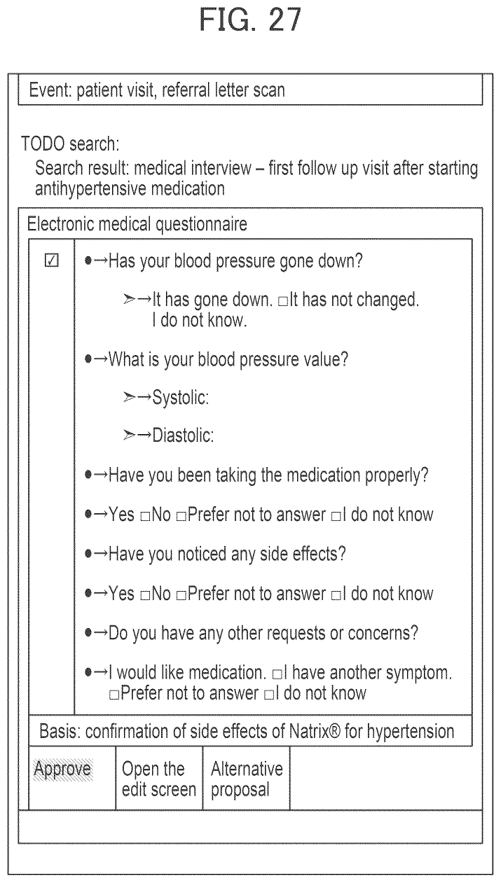

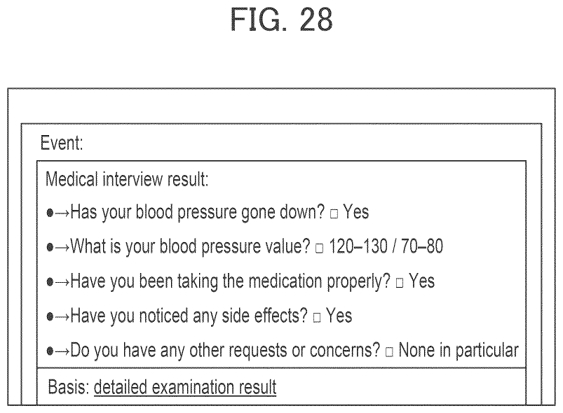

Next, FIGS. 27 and 28 are diagrams showing input results of the electronic medical interview form displayed on the display 241 of the terminal apparatus 20 possessed by the healthcare professional. In FIGS. 27 and 28, the shaded portions indicate options selected and input by the physician using the terminal apparatus 20.

### 5. Effects of One Embodiment

As described in detail above, according to the system 1 of the present embodiment, automation of processing of medical information by healthcare professionals can be achieved.

### 6. Modifications

As a modification, there is a case where both a function 1 in which operation instruction data is automatically executed in response to an input, and a function 2 of confirming approval by a physician when executed, are provided, and a function having a flag indicating that confirmation is necessary or unnecessary for a specific input is provided, and function 1 and function 2 are selected. This function makes it possible to skip approval by a user of an operation instruction for specific input data and automatically execute it. This enables effective input when rapid processing is required.

Specifically, suppose that an instruction for “routine blood collection for side effect evaluation” that is less invasive and performed approximately once a year is described by a physician in the medical record as medical history information at the time of the first visit as “hypertension, routine blood collection performed,” and an examination operation instruction performed on that day is given. In response to this, AI tags the examination operation instruction with “hypertension: routine blood collection” to enable searching. When the physician describes in the medical record as medical history information at subsequent visits, “hypertension, routine blood collection performed today,” the operation instruction tagged with “hypertension routine blood collection” in the past may be automatically implemented in a form to be input on that day. At that time, an input from the user that approval is not required from the next time onward may be accepted, and from the next time onward, when the physician describes in the medical record as medical history information, “hypertension, routine blood collection performed today,” the operation instruction may be automatically registered.

As another example, when a physician describes in the medical record “hypertension, routine blood collection to be performed next time,” when the next appointment is made on that day, the operation instruction may be automatically registered as an operation instruction for the scheduled date. In that case, a check function that confirms whether there are similar examinations immediately before may be provided, and a function that prevents similar orders from being automatically ordered multiple times may be implemented.

As another modification, when multiple operation instruction data are obtained from search results, there is an example of a function that integrates them and removes duplicates during integration. As a specific example, a function that integrates operation instruction data for drug prescriptions obtained as search results and removes duplicates is also included.

In one example, when a physician describes in the medical record as medical history information for a patient being seen in an outpatient clinic for hypertension, “Continue regular hypertension treatment, prescribe the same drug as last year's hay fever medication,” prescription operation instruction A that prescribed the current hypertension medication and operation instruction data B that prescribed the hypertension medication and hay fever medication from one year ago simultaneously are integrated, and by deleting the prescription operation instruction data for the hypertension medication from one year ago, it becomes possible to add the operation instruction data for the same hay fever medication as last time to the prescription operation instruction data for the current hypertension medication. This makes it possible to prescribe with the latest dosage and administration even if the dosage and administration of the hypertension medication has changed over the course of one year.

This function may also be linked with the electronic medical interview form. This function can be realized by providing a function to tag prescription operation instruction data in more detail for each drug, and preparing and having a list of tags that may be assigned to drugs in the content of the medical record description and in a separately prepared database.

In one example, disease names of hay fever for Claritin and hypertension for amlodipine are registered in advance in a database as tag candidates, and when a physician describes in the medical record “prescribe medications for hay fever and hypertension” and operation instruction data is created in which “Claritin and amlodipine” are prescribed simultaneously, the operation instruction data for the entire prescription is tagged with “hypertension+hay fever,” and at the same time, “Claritin” is tagged with hay fever and amlodipine is tagged with “hypertension.”

By performing such tagging in advance, when “Continue regular hypertension treatment, prescribe the same drug as last year's hay fever medication” is described one year later, it becomes possible to search for and obtain last year's hay fever medication and create new operation instruction data.

Furthermore, in another example, it is also possible to have a patient input a request for a prescription for hay fever medication in an electronic medical interview form, and if the patient requests a prescription, duplicate the patient's hay fever prescription content from one year ago, integrate it, and create new operation instruction data. These methods make it possible for physicians to directly reflect patient requests in the EMR. Furthermore, when performing integration and duplicate removal, by preferentially removing duplicates of the latest information among drugs with the same tag, it is ensured that prescriptions are made with the latest dosage and administration.

As another modification, when operation instruction data is obtained from search results, in order to modify it, there is an example of a function that additionally searches for operation instruction data of another patient, integrates the results, and removes duplicates during integration. Specifically, this is a prescription when a patient with hypertension and renal dysfunction develops pneumonia.

In one example, when a physician describes in the medical record as medical history information “Continue regular hypertension treatment, prescribe pneumonia set referring to treatment drugs for pneumonia when renal dysfunction was recognized in another patient,” it is required to integrate prescription operation instruction A that prescribed hypertension medication with prescription operation instruction B that prescribed treatment drugs for pneumonia in a patient with renal dysfunction.

For such a prescription set, disease names of “pneumonia, cough” for Medicon, “pneumonia” for azithromycin, “pneumonia, fever” for Calonal, and “pneumonia, phlegm” for Mucosolvan are registered in advance in a database as tag candidates, and for a patient described in the medical record as “implementing pneumonia treatment,” in a prescription for a patient whose blood collection results show renal dysfunction, if “azithromycin, Calonal, Mucosolvan, Medicon” is prescribed, it is possible to tag the entire prescription with “pneumonia renal dysfunction,” and also tag each prescription of “azithromycin, Calonal, Mucosolvan, Medicon” with “pneumonia renal dysfunction.” From the next time onward, in a patient with pneumonia who shows renal dysfunction in examination results, it becomes possible to obtain operation instruction data for pneumonia treatment in a dosage for a patient with renal dysfunction and issue an operation instruction.

### 6. Supplementary Notes

The embodiments described above provide detailed explanations of the configurations in order to clearly explain the present disclosure, and are not necessarily limited to those having all the configurations described.

Furthermore, it is possible to add, delete, or replace a part of the configuration of each embodiment with other configurations.

As an example, in the embodiment described above, modification/addition of electronic medical interview form data was performed by voice recognition, but modification/addition by voice recognition may also be performed for electronic medical interview forms, EMR templates, and EMR data.

Furthermore, some or all of the above configurations, functions, processing units, processing means, and the like may be realized by hardware, for example, by designing them with an integrated circuit or the like. The present disclosure can also be realized by program code of software that realizes the functions of the embodiments. In this case, a storage medium recording the program code is provided to a computer, and a processor included in the computer reads out the program code stored in the storage medium. In this case, the program code itself read from the storage medium realizes the functions of the embodiments described above, and the program code itself and the storage medium storing it constitute the present invention. As a storage medium for supplying such program code, for example, a flexible disk, CD-ROM, DVD-ROM, hard disk, SSD, optical disk, magneto-optical disk, CD-R, magnetic tape, non-volatile memory card, ROM, or the like is used.

Furthermore, the program code that realizes the functions described in the present embodiment can be implemented in a wide range of programming or scripting languages, such as, for example, assembler, C/C++, Perl, Shell, PHP, Java (registered trademark), and the like.

Furthermore, by distributing the program code of software that realizes the functions of the embodiments via a network, it may be stored in storage means such as a hard disk or memory of a computer or in a storage medium such as a CD-RW or CD-R, and a processor included in the computer may read out and execute the program code stored in the storage means or the storage medium.

The matters described in the above embodiments are appended below.

### (Supplementary Note 1)

A program for operating a computer (30) comprising a processor (39) and a memory (35), wherein the memory (35) stores a search database (3027) that stores operation instruction data used for instructing the computer (30) and/or another computer to execute a specific operation, or a program or a machine learning model (3023, 3024) that generates the operation instruction data, and the program causes the processor (39) to execute: a first step (S750) of accepting an input of at least one of medical history information, information input by a patient, a disease name, a symptom name, a problem in a problem list, and a clinical note; and a second step (S852) of searching the search database (3027) using the input accepted in the first step (S750) to obtain operation instruction data for instructing the computer (30) and/or the other computer to execute a specific operation, or inputting the input accepted in the first step (S750) to the program or the machine learning model (3023, 3024) to obtain operation instruction data for instructing the computer (30) and/or the other computer to execute a specific operation.

### (Supplementary Note 2)

The program of Supplementary Note 1, wherein the operation instruction data includes a plurality of individual operation instruction data used for instructing the computer (30) and/or the other computer to execute a specific operation.

### (Supplementary Note 3)

The program of Supplementary Note 1, wherein at least one of the medical history information, the information input by the patient, the disease name, the symptom name, the problem in the problem list, and the clinical note is described in natural language, or natural language processing is performed in a process when searching the search database (3027), or the program or the machine learning model (3023, 3024) performs natural language processing to generate the operation instruction data.

### (Supplementary Note 4)

The program of Supplementary Note 1, wherein the operation instruction data includes at least one of examination order operation instruction data, prescription order operation instruction data, disease name registration operation instruction data, message transmission operation instruction data, RPA operation instruction data, natural language addition operation instruction data, template display operation instruction data, template data input operation instruction data, filtered examination result display operation instruction data, filtered prescription information display operation instruction data, and appointment operation instruction data.

### (Supplementary Note 5)

The program of Supplementary Note 1, wherein at least one of the following information is used as the input of the first step (S750) of accepting an input of at least one of the medical history information, the information input by the patient, the disease name, the symptom name, the problem in the problem list, and the clinical note: a referral letter, a medication record, optical character recognition (OCR) result information of a scanned examination result, an electronic referral letter, an electronic medication record and an electronic examination result, natural-language information input by the patient, information obtained by voice recognition of speech input by the patient, or a clinical note generated by the program or the machine learning model (3023, 3024).

### (Supplementary Note 6)

The program of Supplementary Note 1, wherein the program further causes the processor (39) to execute a third step (S954) of outputting the operation instruction data obtained in the second step (S852) to the computer (30) and/or the other computer, and instructing the computer (30) and/or the other computer to execute a specific operation.

### (Supplementary Note 7)

The program of Supplementary Note 6, wherein the operation instruction data includes a plurality of individual operation instruction data used for instructing the computer (30) and/or the other computer to execute a specific operation, and in the third step (S954), the operation instruction data including the plurality of individual operation instruction data obtained in the second step (S852) is output to the computer (30) and/or the other computer, and the computer (30) and/or the other computer is instructed to execute a series of specific operations based on the plurality of individual operation instruction data.

### (Supplementary Note 8)

The program of Supplementary Note 6, wherein the program further causes the processor (39) to execute: a fourth step (S955) of instructing the computer (30) and/or the other computer to execute a specific operation in the third step (S954) and accepting an output obtained from the computer (30) and/or the other computer; a fifth step of updating and modifying the medical history information or the clinical note based on the output accepted in the fourth step (S955); and a sixth step of searching the database using the medical history information or the clinical note updated in the fifth step as an input to obtain operation instruction data for instructing the computer (30) and/or the other computer to execute a specific operation, or inputting the medical history information or the clinical note updated in the fourth step (S955) to the program or the machine learning model (3023, 3024) to obtain again operation instruction data for instructing the computer (30) and/or the other computer to execute a specific operation.

### (Supplementary Note 9)

The program of Supplementary Note 1, wherein the program further causes the processor (39) to execute a seventh step (S953) of accepting an approval input as to whether or not to instruct the computer (30) and/or the other computer to execute a specific operation based on the operation instruction data obtained in the second step (S852).

### (Supplementary Note 10)

The program of Supplementary Note 9, wherein in the seventh step (S953), information indicating a summary of the operation instruction data obtained in the second step (S852) is generated and presented, and after presenting the information, an approval input as to whether or not to instruct the computer (30) and/or the other computer to execute a specific operation based on the operation instruction data is accepted.

### (Supplementary Note 11)

The program of Supplementary Note 8, wherein the program further causes the processor (39) to execute an eighth step (S954) of presenting update and modification content and accepting approval of the update when updating the medical history information or the clinical note based on the output obtained in the fourth step (S955).

### (Supplementary Note 12)

The program of Supplementary Note 7, wherein in the seventh step (S953), when the obtained operation instruction data includes a plurality of options, a selection input of at least one of the plurality of options is accepted.

### (Supplementary Note 13)

The program of Supplementary Note 11, wherein in the eighth step (S954), when the update and modification of the medical history information or the clinical note includes a plurality of options, a selection input of at least one of the plurality of options is accepted.

### (Supplementary Note 14)

The program of Supplementary Note 9, wherein the program further causes the processor (39) to execute a ninth step of outputting the operation instruction data for which the approval input was made in the seventh step (S953) to the computer (30) and/or the other computer, and instructing the computer (30) and/or the other computer to execute a specific operation.

### (Supplementary Note 15)

The program of Supplementary Note 1, wherein the search database (3027) has, as a search key, a word included in at least one of the medical history information, the information input by the patient, the disease name, the symptom name, the problem in the problem list, and the clinical note, or a tag created by performing natural language processing based on at least one of the medical history information, the information input by the patient, the disease name, the symptom name, the problem in the problem list, and the clinical note, and the operation instruction data for instructing the computer (30) and/or the other computer to execute a specific operation is associated with the tag.

### (Supplementary Note 16)

The program of Supplementary Note 15, wherein the search database (3027) includes a tag obtained by converting date information such as a hospitalization start date, a surgery date, and a first outpatient visit date included in the medical history information, the information input by the patient, the disease name, the symptom name, the problem in the problem list, and the clinical note into a relative date and time from a predetermined date and time by natural language processing.

### (Supplementary Note 17)

The program of Supplementary Note 16, wherein the program further causes the processor (39) to execute: a tenth step of generating a tag from a word included in at least one of the medical history information, the information input by the patient, the disease name, the symptom name, the problem in the problem list, and the clinical note, or performing natural language processing based on the medical history information, the information input by the patient, the disease name, the symptom name, the problem in the problem list, and the clinical note to generate a tag; and an eleventh step of presenting at least one of the medical history information, the information input by the patient, the disease name, the symptom name, the problem in the problem list, and the clinical note that is a basis for generating the tag when operation instruction data based on the tag is searched, wherein in the tenth step, a link to the medical history information and a position of a disease name included in the medical history information are recorded in the search database (3027), and wherein in the eleventh step, when presenting at least one of the medical history information, the information input by the patient, the disease name, the symptom name, the problem in the problem list, and the clinical note that is the basis for generating the tag, if at least one of the medical history information, the information input by the patient, the disease name, the symptom name, the problem in the problem list, and the clinical note that is the basis for generating the tag is presented for the first time, the fact that it is presented for the first time is presented, and when there are a plurality of medical history information, information input by the patient, disease names, symptom names, problems in the problem list, and clinical notes that are the basis for generating the tag, the medical history information, the information input by the patient, the disease name, the symptom name, the problem in the problem list, and the clinical note that is the basis for generating the tag having the oldest date information is presented.

### (Supplementary Note 18)

The program of Supplementary Note 15, wherein the memory (35) stores a medical standard glossary, and the program further causes the processor (39) to execute a twelfth step of: searching for synonyms based on a word included in at least one of the medical history information, the information input by the patient, the disease name, the symptom name, the problem in the problem list, and the clinical note, or a word obtained by performing natural language processing on at least one of the medical history information, the information input by the patient, the disease name, the symptom name, the problem in the problem list, and the clinical note; or searching for a cosine similarity of the medical history information and the like to obtain a word in the standard glossary, and setting the word as a new tag in the search database (3027).

### (Supplementary Note 19)

The program of Supplementary Note 1, wherein the program further causes the processor (39) to execute a thirteenth step of accepting a modification input for a search tag of the search database (3027) from a user, and modifying the search tag based on the modification input.

### (Supplementary Note 20)

The program of Supplementary Note 1, wherein the search database (3027) stores a comment for a search tag and/or information indicating a deprecated state in association with the tag, and in the second step (S852), when the search database (3027) is searched based on the tag, if the comment is associated with the tag, the comment is presented, and if the information indicating the deprecated state is associated with the tag, a presentation manner of the operation instruction data associated with the tag is made different from a presentation manner of other operation instruction data.

### (Supplementary Note 21)

The program of Supplementary Note 15, wherein the search database (3027) stores information indicating a flag input for a tag and/or a number of positive and negative feedbacks in association with the tag, and in the second step (S852), when searching the search database (3027), if the flag is associated with the tag, the operation instruction data is filtered or preferentially displayed based on the flag, and if information indicating one of the positive feedback and the negative feedback is associated with the tag, and if the number of negative feedbacks is greater than the positive feedbacks, the operation instruction data is preferentially displayed.

### (Supplementary Note 22)

The program of Supplementary Note 7, wherein the search database (3027) stores, in association with the operation instruction data, a number of operation instructions of the operation instruction data for each user, a number of operation instructions of the operation instruction data for each department to which the user belongs, a name of a patient related to the operation instruction data, an age of the patient, a creator of the operation instruction data, a job title of the creator, or a department to which the creator belongs, and in the second step (S852), a display manner of the operation instruction data is changed based on at least one of: a number of past operation instructions of the user, a number of operation instructions by department, a number of operation instructions at a medical institution, the name of the patient, the age of the patient, the creator of the operation instruction data, the job title of the creator of the operation instruction data, the department to which the creator belongs, a number of execution instructions of the operation instruction data based on the operation instruction data in the department to which the user belongs, a ranking of the number of execution instructions based on the operation instruction data for each job title, or a ranking of a number of individual operation instruction data associated with the operation instruction data.

### (Supplementary Note 23)

The program of Supplementary Note 1, wherein in the second step (S852), an utterance input from a user is accepted, and the search database (3027) is searched based on the utterance input to obtain the operation instruction data.

### (Supplementary Note 24)

The program of Supplementary Note 1, wherein in the second step (S852), an utterance input from a user is accepted, and the obtained operation instruction data is modified based on the utterance input.

### (Supplementary Note 25)

The program of Supplementary Note 19, wherein in the second step (S852), for the operation instruction data obtained by searching the search database (3027) with the search tag modified in the thirteenth step, an utterance input from the user is accepted, and the operation instruction data is modified based on the utterance input.

### (Supplementary Note 26)

The program of Supplementary Note 1, wherein the machine learning model (3023, 3024) is created by performing machine learning or prompt engineering based on the tag and the operation instruction data.

### (Supplementary Note 27)

The program of Supplementary Note 15, wherein a plurality of tags are provided in the search database (3027), and in the second step (S852), when searching the search database (3027), whether or not a tag is included in at least one of the medical history information, the information input by the patient, the disease name, the symptom name, the problem in the problem list, and the clinical note is searched, and operation instruction data corresponding to the tag for instructing the computer (30) and/or the other computer to execute a specific operation is obtained as a search result of the search database (3027).

### (Supplementary Note 28)

The program of Supplementary Note 1, wherein the memory (35) stores at least one of past medical history information, information input by the patient, a disease name, a symptom name, a problem in a problem list, and a clinical note, an input date and time of the medical history information and the like, past operation instruction data, and a generation date and time of the operation instruction data, and the program further causes the processor (39) to execute a fourteenth step of generating operation instruction data of the search database (3027) based on operation instruction data for which an operation instruction was executed simultaneously with, on the same day as, in the same month as, or in association with an input of at least one of the past medical history information, the information input by the patient, the disease name, the symptom name, the problem in the problem list, and the clinical note or generation of a tag.

### (Supplementary Note 29)

The program of Supplementary Note 28, wherein in the fourteenth step, an identifier included in at least one of the past medical history information, the information input by the patient, the disease name, the symptom name, the problem in the problem list, and the clinical note and a word following the identifier are extracted, and the extracted word is set as a tag.

### (Supplementary Note 30)

The program of Supplementary Note 29, wherein the identifier is an identifier included in the past problem list, and in the fourteenth step, the disease name and the symptom name included in the problem list are set as a tag.

### (Supplementary Note 31)

The program of Supplementary Note 28, wherein at least one of the past medical history information, the information input by the patient, the disease name, the symptom name, the problem in the problem list, and the clinical note includes past examination information, and in the second step (S852), examination order operation instruction data is obtained as the operation instruction data, and the examination operation instruction data that is the same as or synonymous with the past examination information is displayed.

### (Supplementary Note 32)

The program of Supplementary Note 28, wherein at least one of the past medical history information, the information input by the patient, the disease name, the symptom name, the problem in the problem list, and the clinical note includes past prescription information, and in the second step (S852), prescription order operation instruction data is obtained as the operation instruction data, and a medication having the same ingredient as the past prescription information is displayed in a filtered manner, highlighted, or displayed at a higher rank.

### (Supplementary Note 33)

The program of Supplementary Note 28, wherein at least one of the past medical history information, the information input by the patient, the disease name, the symptom name, the problem in the problem list, and the clinical note includes a disease name of a past medical history and the like, and in the second step (S852), disease name registration operation instruction data is obtained as the operation instruction data, the disease name registration operation instruction data that is the same as or synonymous with the disease name is confirmed, and a disease name that is not registered is displayed in a filtered manner, highlighted, or displayed at a higher rank.

### (Supplementary Note 34)

The program of Supplementary Note 28, wherein at least one of the past medical history information, the information input by the patient, the disease name, the symptom name, the problem in the problem list, and the clinical note includes a disease name of a past medical history and the like, and in the second step (S852), disease name registration operation instruction data is obtained as the operation instruction data, and an input of a secondary disease name of the disease name is accepted.

### (Supplementary Note 35)

The program of Supplementary Note 1, wherein the program causes the processor (39) to execute a fifteenth step of accepting an instruction input by natural language or an option button, and obtains the operation instruction data by using, as the input of the second step (S852), at least one of the medical history information, the information input by the patient, the disease name, the symptom name, the problem in the problem list, and the clinical note, and the instruction input.

### (Supplementary Note 36)

The program of Supplementary Note 1, wherein the program further causes the processor (39) to execute a sixteenth step of presenting, in a simultaneously viewable state, a search result of the search database (3027) or an output result of a program or a machine learning model (3023, 3024) that generates the operation instruction data, and the operation instruction data for instructing the computer (30) and/or the other computer to execute a specific operation.

### (Supplementary Note 37)

The program of Supplementary Note 1, wherein the program further causes the processor (39) to execute a seventeenth step of: generating information summarizing at least one of the medical history information, the information input by the patient, the disease name, the symptom name, the problem in the problem list, and the clinical note, and natural language indicating a reason for searching for the operation instruction data to the computer (30) and/or the other computer; presenting the medical history information and the like, the information summarizing the medical history information and the like, and the natural language; and presenting an approval button or a selection button for the operation instruction data in a simultaneously viewable state.

### (Supplementary Note 38)

A program for operating a computer (30) comprising a processor (39) and a memory (35), wherein the memory (35) stores data in which at least medical history or data generated from the medical history and medical instruction information or medical history addition instruction information are stored in pairs, and the program causes the processor (39) to execute: an eighteenth step of accepting an input of medical history; a nineteenth step of generating a search word based on the medical history accepted in the eighteenth step; and a twentieth step of searching for medical instruction information based on the search word generated in the nineteenth step and presenting the medical instruction information to a user.

### (Supplementary Note 39)

The program of Supplementary Note 37, wherein the program further causes the processor (39) to execute a twenty-first step of displaying a frequency of the medical instruction information or a user name or a job title of a user who made a medical instruction in the past.

### (Supplementary Note 40)

An information processing apparatus comprising a processor (39) and a memory (35), wherein the memory (35) stores a search database (3027) that stores operation instruction data used for instructing the computer (30) and/or another computer to execute a specific operation, or a program or a machine learning model (3023, 3024) that generates the operation instruction data, and the processor (39) executes: a first step (S750) of accepting an input of at least one of medical history information, information input by a patient, a disease name, a symptom name, a problem in a problem list, and a clinical note; and a second step (S852) of searching the search database (3027) using the input accepted in the first step (S750) to obtain operation instruction data for instructing the computer (30) and/or the other computer to execute a specific operation, or inputting the input accepted in the first step (S750) to the program or the machine learning model (3023, 3024) to obtain operation instruction data for instructing the computer (30) and/or the other computer to execute a specific operation.

### (Supplementary Note 41)

An information processing apparatus comprising a processor (39) and a memory (35), wherein the memory (35) stores data in which at least medical history or data generated from the medical history and medical instruction information or medical history addition instruction information are stored in pairs, and the processor (39) executes: an eighteenth step of accepting an input of medical history; a nineteenth step of generating a search word based on the medical history accepted in the eighteenth step; and a twentieth step of searching for medical instruction information based on the search word generated in the nineteenth step and presenting the medical instruction information to a user.

### (Supplementary Note 42)

A method executed by a computer (30) comprising a processor (39) and a memory (35), wherein the memory (35) stores a search database (3027) that stores operation instruction data used for instructing the computer (30) and/or another computer to execute a specific operation, or a program or a machine learning model (3023, 3024) that generates the operation instruction data, and the processor (39) executes: a first step (S750) of accepting an input of at least one of medical history information, information input by a patient, a disease name, a symptom name, a problem in a problem list, and a clinical note; and a second step (S852) of searching the search database (3027) using the input accepted in the first step (S750) to obtain operation instruction data for instructing the computer (30) and/or the other computer to execute a specific operation, or inputting the input accepted in the first step (S750) to the program or the machine learning model (3023, 3024) to obtain operation instruction data for instructing the computer (30) and/or the other computer to execute a specific operation.

### (Supplementary Note 43)

A method executed by a computer (30) comprising a processor (39) and a memory (35), wherein the memory (35) stores data in which at least medical history or data generated from the medical history and medical instruction information or medical history addition instruction information are stored in pairs, and the processor (39) executes: an eighteenth step of accepting an input of medical history; a nineteenth step of generating a search word based on the medical history accepted in the eighteenth step; and a twentieth step of searching for medical instruction information based on the search word generated in the nineteenth step and presenting the medical instruction information to a user.

### (Supplementary Note 44)

A system comprising: a memory (35) that stores a search database (3027) that stores operation instruction data used for instructing a computer (30) and/or another computer to execute a specific operation, or a program or a machine learning model (3023, 3024) that generates the operation instruction data; means for accepting an input of at least one of medical history information, information input by a patient, a disease name, a symptom name, a problem in a problem list, and a clinical note; and means for searching the search database (3027) using the input accepted by the means for accepting the input of the medical history information to obtain operation instruction data for instructing the computer (30) and/or the other computer to execute a specific operation, or inputting the input accepted in the first step (S750) to the program or the machine learning model (3023, 3024) to obtain operation instruction data for instructing the computer (30) and/or the other computer to execute a specific operation.

### (Supplementary Note 45)

A system comprising: a memory (35) that stores data in which at least medical history or data generated from the medical history and medical instruction information or medical history addition instruction information are stored in pairs; means for accepting an input of medical history; means for generating a search word based on the medical history accepted by the means for accepting the input of medical history; and means for searching for medical instruction information based on the search word generated by the means for generating the search word and presenting the medical instruction information to a user.

What is claimed is:

1. An information processing apparatus comprising:
a processor; and
a memory storing a search database in which operation instruction data used to instruct the information processing apparatus and/or another information processing apparatus to execute a specific operation is stored,
wherein the search database stores, in association with at least one word or sentence included in at least one of:
medical history information;
information input by a patient;
a disease name;
a symptom name; or
a problem in a problem list,
one of:
(i) operation instruction data for instructing the information processing apparatus and/or the other information processing apparatus to execute the specific operation; or
(ii) a search word or a search sentence used for searching the operation instruction data,
and wherein the processor is configured to:
receive, as an input, at least one of:
the medical history information;
the information input by the patient;
the disease name;
the symptom name; or
the problem in the problem list;
search the search database using the input and obtain, as the operation instruction data, operation instruction data corresponding to a word or a sentence included in the input;
present, on a display, the obtained operation instruction data as at least one candidate to a healthcare professional and receive an approval input from the healthcare professional; and
in response to receiving the approval input, output the operation instruction data to the information processing apparatus and/or the other information processing apparatus so as to cause execution of the specific operation.

2. An information processing apparatus comprising:
a processor; and
a memory storing a generation program configured, on the basis of at least one word or sentence included in at least one of:
medical history information;
information input by a patient;
a disease name;
a symptom name;
a problem in a problem list; or
a clinical note,
to generate operation instruction data used to instruct the information processing apparatus and/or another information processing apparatus to execute a specific operation,
wherein the processor is configured to:
receive, as an input, at least one of:
the medical history information;
the information input by the patient;
the disease name;
the symptom name;
the problem in the problem list; or
the clinical note;
provide the received input to the generation program to generate the operation instruction data;
present, on a display, the generated operation instruction data as at least one candidate to a healthcare professional and receive an approval input from the healthcare professional; and
in response to receiving the approval input, output the operation instruction data to the information processing apparatus and/or the other information processing apparatus so as to cause execution of the specific operation.

3. A computer-implemented method executed by a computer including a processor and a memory,
the method comprising:
maintaining, in the memory, structured data of an electronic medical record (EMR) template, the structured data associating input items of the EMR template with input content;
maintaining, in the memory, a search database in which operation instruction data used to instruct the computer and/or another computer to execute a specific medical operation is stored;
receiving, as an input, at least one of:
medical history information;
information input by a patient;
a disease name;
a symptom name; or
a problem in a problem list;
specifying, on the basis of the EMR template and the received input, at least one input item of the EMR template and input content corresponding to the input item;
updating EMR template data by recording the input content in association with the specified input item of the EMR template;
searching the search database using at least one of the input and the updated EMR template data and obtaining, as operation instruction data, operation instruction data corresponding to clinical information recorded in an EMR represented by the input;
presenting the obtained operation instruction data as at least one candidate to a healthcare professional and receiving an approval input from the healthcare professional;
in response to receiving the approval input, outputting the operation instruction data to the computer and/or the other computer so as to cause execution of the specific medical operation; and
in response to execution of the specific medical operation by the computer and/or the other computer, receiving result data and updating at least one of:
the medical history information; or
a clinical note,
on the basis of the result data.

4. The information processing apparatus according to claim 1,
wherein the operation instruction data includes a plurality of individual operation instruction data items used to instruct the information processing apparatus and/or the other information processing apparatus to execute a plurality of operations.

5. The information processing apparatus according to claim 1,
wherein at least one of:
the medical history information;
the information input by the patient;
the disease name;
the symptom name; or
the problem in the problem list is described in natural language,
and/or the processor performs natural language processing when searching the search database.

6. The information processing apparatus according to claim 1,
wherein the processor is configured to receive, as at least part of the input, at least two combinations of:
a referral letter;
a medication record;
optical character recognition result information of a scanned examination result;
an electronic referral letter;
an electronic medication record;
an electronic examination result;
natural-language information input by the patient;
speech input by the patient and text information obtained by converting the speech input into text by speech recognition; and
clinical notes generated by another program or a machine learning model.

7. The information processing apparatus according to claim 1,
wherein the operation instruction data includes at least one of:
examination order operation instruction data; or
prescription order operation instruction data.

8. The information processing apparatus according to claim 7,
wherein past medical history information includes past examination information,
and wherein, when the operation instruction data includes examination order operation instruction data,
the processor is configured to obtain examination order operation instruction data as the operation instruction data and display, among the examination order operation instruction data, examination order operation instruction data that is the same as or synonymous with the past examination information.

9. The information processing apparatus according to claim 7,
wherein past medical history information includes past prescription information,
and wherein, when the operation instruction data includes prescription order operation instruction data, the processor is configured to obtain prescription order operation instruction data as the operation instruction data and, among the prescription order operation instruction data, to preferentially display prescription order operation instruction data relating to drugs having the same active ingredient as drugs included in the past prescription information.

10. The information processing apparatus according to claim 1,
further comprising an output interface,
wherein the processor is further configured to:
output the operation instruction data, in response to receiving the approval input, to the information processing apparatus and/or the other information processing apparatus via the output interface so as to instruct execution of the specific operation; and
receive, from the information processing apparatus and/or the other information processing apparatus, an output generated in response to execution of the specific operation and update at least one of:
the medical history information; or
a clinical note,
on the basis of the output.

11. The information processing apparatus according to claim 2,
wherein the generation program includes a machine learning model that is created by machine learning or prompt engineering on the basis of pairs of:
(i) at least one of medical history information, information input by a patient, a disease name, a symptom name, a problem in a problem list, or a clinical note, or tags created from at least one of them by natural language processing; and
(ii) operation instruction data.

13. The information processing apparatus according to claim 2,
wherein the operation instruction data generated by the generation program includes at least one of:
examination order operation instruction data; or
prescription order operation instruction data.

14. The method according to claim 3,
wherein the operation instruction data includes at least one of:
examination order operation instruction data; or
prescription order operation instruction data, and the method further comprises displaying, as candidates, at least one examination order and/or prescription order on the basis of the operation instruction data.

15. The method according to claim 3, further comprising:
outputting the operation instruction data to the computer and/or another computer and thereby instructing the computer and/or the other computer to execute the specific operation in response to receiving the approval input;
receiving an output generated by the computer and/or the other computer in response to execution of the specific operation; and
updating at least one of:
medical history information; or
a clinical note,
on the basis of the output.

16. The information processing apparatus according to claim 1,
wherein, when the operation instruction data includes instruction to generate a document,
and the processor is further configured to:
obtain structured data including at least one of:
content of an EMR template; or
laboratory test results from an EMR;
generate, using a machine learning model based on EMR data, a template text having a slot for inserting the structured data; and
insert the obtained structured data into the slot of the template text to generate the document.
